(19) United States
(12) Patent Application Publication
(10) Pub. No.: US 2004/0142892 A1
(43) Pub. Date: Jul. 22, 2004

(54) Autogene nucleic acids encoding a secretable RNA polymerase

(75) Inventors: John Finn, Vancouver (CA); Ian MacLachlan, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The University of British Columbia, Vancouver (CA)

(21) Appl. No.: 10/688,299

(22) Filed: Oct. 16, 2003

Related U.S. Application Data
(63) Continuation-in-part of application No. 10/136,738, filed on Apr. 30, 2002.
(60) Provisional application No. 60/287,974, filed on Apr. 30, 2001.

(51) Int. Cl. A61K 48/00; C07H 21/04; C12N 9/22

(52) U.S. Cl. 514/44; 435/69.1; 435/199; 435/320.1; 435/325; 536/23.2

(57) ABSTRACT

This invention provides methods, nucleic acids, compounds, and compositions for expressing a product of interest in a cell that involve a secretable RNA Polymerase.

### CROSS-REFERENCES TO RELATED APPLICATIONS

This application is a continuation in part of U.S. patent application Ser. No. 10/136,738, filed Apr. 30, 2002, which claims the benefit of U.S. patent application Ser. No. 60/287,974, filed Apr. 30, 2001, the disclosures of which are hereby incorporated by reference in their entirety for all purposes.

### BACKGROUND OF THE INVENTION

Recombinant DNA methods permit the construction of nucleic acid eukaryotic expression cassettes encoding a product of interest. These expression cassettes are then introduced into the cytoplasm of eukaryotic cells using methods known in the art. However, a major difficulty in the expression of these expression cassettes is that the nucleic acid encoding the product of interest must be exported into the nucleus where the eukaryotic transcription machinery resides. Those expression cassettes that remain in the cytoplasm are not transcribed due to the lack of a cytoplasmic RNA polymerase that can transcribe the cassette.

One strategy to increase levels of expression of the product of interest from expression cassettes following non-viral cell transfection involves the use of a cytoplasmic expression system (Gao and Huang (1993) Nucleic Acids Res. 21: 2867-2872). The advantage of such a system is that it bypasses the need for nuclear delivery of plasmid DNA, a major obstacle in present day expression systems and in gene therapy. The efficiency of nuclear delivery following intracellular delivery is very low and is dependent on the size of the plasmid DNA molecule (Hagstrom et al. (1997) J Cell Sci. 110: 2323-2331). The addition of nuclear localization signals to plasmid DNA, has been shown to enhance transfection, but with limited success (Arohsohn and Hughes (1998) J Drug Targeting 5: 163-169). The primary barrier to nuclear delivery of plasmid DNA is thought to be the nuclear membrane as plasmid DNA enters the nucleus more efficiently in mitotic or dividing cells, during the breakdown of the nuclear envelope (Coonrod et al. (1997) Gene Ther. 4: 1313-1321). As a result, gene expression following transfection is much higher in dividing than non-dividing cells (Vitadelo et al. (1994) Hum. Gen. Ther. 5: 11-18; Miller et al., (1992) Mol. Cell. Biol. 10: 4239-4242). A further limitation of nuclear expression systems is the finite, saturable limit to the amount of DNA that can be taken up by the nucleus under any condition (Brisson et al. (1999) Human Gene Therapy 10: 2601-2613).

A major limitation of gene delivery systems is the relatively low level of gene expression in transfected tissues. One strategy to increase levels of gene expression following transfection employing a non-viral vector involves improving the plasmid design. The incorporation of a cytoplasmic expression system represents one such approach (see, e.g., Gao and Huang Nucleic Acids Res. 21(12):2867-2872 (1993); Elroy-Stein and Moss PNAS USA 87(17):6743-7 (1990); and Dubendorff and Studier J. Mol. Biol. 219(1):61-8 (1991)). Cytoplasmic expression systems bypass the requirement for nuclear delivery of plasmid DNA, a major obstacle in present day gene therapy (see, e.g., Capecchi Cell 22(2 Pt 2):479-88 (1980); Zabner, et al. J. Biol. Chem. 270:18997-19007 (1995); Wilke, et al. Gene Ther. 3(12):1133-42 (1996); and Coonrod, et al. Gene Ther. 4(12):1313-21 (1997)). In addition, they take advantage of the large number of plasmids found in the cytoplasm of the cell following transfection with non-viral vectors (see, e.g., Lechardeur, et al. Gene Ther. 6:492-497 (1999)). Cytoplasmic expression systems can be designed to utilize the unique properties of the bacteriophage RNA polymerases (RNAPs). Phage RNAPs are moderately sized (˜100 kD), single subunit proteins capable of synthesizing RNA from DNA templates. They require no additional co-factors and have demonstrated efficient cytoplasmic transcriptional activity (see, e.g., Chamberlin, et al., Nature 228(268):227-31 (1970) and Dunn, et al. Nat. New Biol. 230(11):94-6 (1971)). These features make phage RNAPs attractive candidates for the development of autocatalytic cytoplasmic expression systems using autogenes. Phage RNAP autogenes consist of an RNAP gene, driven by its own cognate promoter (see, e.g., . Dubendorff and Studier J. Mol. Biol. 219(1):61-8 (1991)). In order to evade the requirement for exogenous RNAP to initiate the expression system, a nuclear promoter can be added upstream of the RNAP promoter (see, e.g., Brisson, et al. Gene Ther. 6(2):263-270 (1999)). Although the first round of RNAP expression must occur via the nuclear promoter, the resulting RNAP in the cytoplasm drives the cytoplasmic expression system, producing RNA from plasmid DNA template in the cytoplasm.

RNA produced in the cytoplasm lacks the 5′ cap that stabilizes nuclear transcripts and assists in ribosomal recruitment (see, e.g., Kaempfer, et al. PNAS USA 75(2):650-4 (1978) and Drummond, et al. Nucleic Acids Res. 13(20):7375-94 (1985)). Viral Internal Ribosome Entry Site (IRES) elements are sequences that have been shown to enhance the recruitment of the cytoplasmic translational machinery in the absence of 5′ capping (see, e.g., Jang and Wimmer Genes Dev. 4(9):1560-72 (1990)). Early dual promoter cytoplasmic expression systems did not contain IRES elements, and as a result, the vast majority of the mRNA produced was not translated (see, e.g., Brisson, et al. Gene Ther. 6(2):263-270 (1999)). Although an autogene based on the T7 bacteriophage RNAP that contained an EMCV IRES has been previously described (see, e.g., Deng, et al. Gene 143(2):245-9 (1994)), it did not contain a eukaryotic promoter and required the co-transfection of RNAP protein or mRNA, thereby limiting its utility.

Attempts have been made to incorporate non-host RNA polymerase promoters and genes encoding RNA polymerases with expression systems to overcome the above limitations. More particularly, these limitations have led to the development of strategies that do not require nuclear localization of DNA. One of these involves the use of bacteriophage T7 RNA polymerase (T7 RNAP). T7 RNAP is a single polypeptide enzyme that mediates transcription in the cytoplasm with high promoter specificity and efficiency (Davanloo et al. (1984) Proc. Natl. Acad. Sci., U.S.A. 81: 2035-2039). These properties have facilitated the development of a T7 based cytoplasmic expression system. Such systems require cytoplasmic delivery of both a plasmid construct containing a gene of interest under transcriptional control of the T7 promoter and a source of the T7 polymerase. Initial studies involved co-transfection of cells with plasmids carrying T7 controlled genes and purified T7 RNAP protein. These systems were able to bypass the need for the nuclear transcription machinery and yielded high levels of gene expression (Gao and Huang (1993)). Due to the instability of the T7 RNAP protein, however, the resulting gene expression was short lived, and considerable T7 RNAP associated cytotoxicity was observed (Gao and Huang (1993)).

These studies led to the development of the T7 polymerase autogene. This system consists of a T7 RNAP gene driven by its own T7 promoter, along with a reporter gene, on different plasmids. When cells were co-transfected with these constructs and purified T7 RNAP protein, rapid and sustained levels of reporter protein were detected. The T7 autogene was able to replenish its supply of T7 RNAP, resulting in sustained gene expression (Chen et al. (1994) Nucleic Acids Res. 22: 2114-2120). While these autogenes are effective, the transfection cocktail is difficult to prepare and, in practice, has been shown to be cytotoxic. To overcome these problems, a dual promoter autogene was created (Brisson et al. (1999) Gene Ther. 6: 263-270). This construct contained a T7 RNAP gene in control of both T7 (cytoplasmic) and CMV (nuclear) promoters. This construct when taken up into the nucleus resulted in low levels of T7 RNAP being produced. The T7 RNAP produced in the nucleus in turn is able to transcribe the cytoplasmic plasmid, which is the major portion of plasmid in the cell. This in turn leads to more T7 RNAP being produced which acts to amplify the production of more T7 RNAP and the reporter gene product. Theoretically, one plasmid incorporated into the nucleus would be sufficient to activate and induce high levels of gene expression from thousands of cytoplasmic plasmids. However, this effect is limited to the cell in which the RNAP is being expressed. Other cells in which DNA is not being expressed in the nucleus, do not show the autogene effect.

Thus, a need exists in the art for nucleic acids, nucleic acid compositions, and methods that permit a RNAP to enter a cell containing cytoplasmic expression cassettes and to express the nucleic acid in the cassette that is under the control of a RNA polymerase promoter. The present invention fulfills these and other needs in the art.

### SUMMARY OF THE INVENTION

The present invention provides nucleic acids encoding a secretable RNA polymerase (sRNAP) containing a RNA polymerase (RNAP) linked to a secretion domain (i.e., an autogene construct), compositions comprising such nucleic acids, and methods of using such nucleic acids and compositions.

One embodiment of the present invention is a nucleic acid (i.e., a vector) comprising a secretable RNA polymerase expression cassette. The expression cassette comprises (1) a eukaryotic promoter and a RNA polymerase promoter operably linked to a nucleic acid encoding a secretable RNA polymerase comprising a RNA polymerase, a secretion domain, and a first internal ribosome entry site; and (2) a RNA polymerase promoter operably linked to a nucleic acid encoding a product of interest and a second internal ribosome entry site. One aspect of the invention provides a host cell comprising the vector comprising the expression cassette described herein.

In certain embodiments, the RNA polymerase is a non-host RNA polymerase. Examples of RNAPs that can be linked to a secretion domain include, but are not limited to, a phagemid RNA polymerase, a prokaryotic RNA polymerase, an archaebacterial RNA polymerase, a plant RNA polymerase, a fungal RNA polymerase, a eukaryotic RNA polymerase, a viral RNA polymerase, mitochondrial RNA polymerase, and a chloroplast RNA polymerase. In particularly preferred embodiments, the RNAPs are selected from the group consisting of a SP6 RNA Polymerase, a T7 RNA Polymerase, a K11 RNA Polymerase, and a T3 RNA Polymerase.

The secretion domains that are linked to the RNAP can be synthesized or obtained from any of a variety of different sources. For example, the secretion domains can be chosen from the following secretion domains:

SEQ ID NO: 1 (HIV-Tat, Tyr-Gly-Arg-Lys-Lys-Arg-
Arg-Gln-Arg-Arg-Arg); SEQ ID NO: 2 (HIV-Tat Variant, Tyr-Ala-Arg-Lys-Ala-Arg-Arg-
Gln-Ala-Arg-Arg); SEQ ID NO: 3 (HIV-Tat Variant, Tyr-Ala-Arg-Ala-Ala-Ala-Arg-Gln-
Ala-Arg-Ala); SEQ ID NO: 4 (HIV-Tat Variant, Tyr-Ala-Arg-Ala-Ala-Arg-Ala-Ala-Arg-
Arg-Arg); SEQ ID NO: 5 (HIV-Tat Variant, Tyr-Ala-Arg-Ala-Ala-Arg-Ala-Ala-Arg-Arg-
Ala); SEQ ID NO: 6 (HIV-Tat Variant, Tyr-Ala-Arg-Arg-Arg-Arg-Arg-Arg-Arg-Arg-Arg);
SEQ ID NO: 7 (HIV-Tat Variant, Tyr-Ala-Ala-Ala-Ala-Arg-Arg-Arg-Arg-Arg-Arg); SEQ
ID NO: 8 (HIV-Tat Variant, Ala-Gly-Arg-Lys-Lys-Arg-Arg-Gln-Arg-Arg-Arg); SEQ ID
NO: 9 (HSV VP22, Asp-Ala-Ala-Thr-Ala-Thr-Arg-Gly-Arg-Ser-Ala-Ala-Ser-Arg-Pro-Thr-
Glu-Arg-Pro-Arg-Ala-Pro-Ala-Arg-Ser-Ala-Ser-Arg-Pro-Arg-Arg-Pro-Val-Glu); SEQ ID
NO: 10 (Antennapedia third Helix, 43-58, Penetratin-1, Arg-Gln-Ile-Lys-Ile-Trp-Phe-Gln-
Asn-Arg-Arg-Met-Lys-Trp-Lys-Lys); SEQ ID NO: 11 (Antennapedia third Helix, 53-43,
Lys-Lys-Trp-Lys-Met-Arg-Arg-Asn-Gln-Phe-Trp-Ile-Lys-Ile-Gln-Arg); SEQ ID NO: 12
(Antennapedia third Helix, 43-58, D-amino acids Arg-Gln-Ile-Lys-Ile-Trp-Phe-Gln-Asn-Arg-
Arg-Met-Lys-Trp-Lys-Lys); SEQ ID NO: 13 (Antennapedia third Helix, 43-58, Pro50, Arg-
Gln-Ile-Lys-Ile-Trp-Phe-Pro-Asn-Arg-Arg-Met-Lys-Trp-Lys-Lys); SEQ ID NO: 14
(Antennapedia third Helix, 43-58, 3-Pro, Arg-Gln-Pro-Lys-Ile-Trp-Phe-Pro-Asn-Arg-Arg-
Lys-Pro-Trp-Lys-Lys); SEQ ID NO: 15 (Antennapedia third Helix, 43-58, R52M/M54R,
Arg-Gln-Ile-Lys-Ile-Trp-Phe-Gln-Asn-Met-Arg-Arg-Lys-Trp-Lys-Lys); SEQ ID NO: 16
(Antennapedia third Helix, 43-58, 7-Arg, Arg-Gln-Ile-Arg-Ile-Trp-Phe-Gln-Asn-Arg-Arg-
Met-Arg-Trp-Arg-Arg); SEQ ID NO: 17 (Antennapedia third Helix, 43-58, W/R, Arg-Arg-
Trp-Arg-Arg-Trp-Trp-Arg-Arg-Trp-Trp-Arg-Arg-Trp-Arg-Arg); SEQ ID NO: 18 (Kaposi's
FGF signal sequence, truncated Ala-Ala-Val-Ala-Leu-Leu-Pro-Ala-Val-Leu-Leu-Ala-Leu-
Leu-Ala-Pro); SEQ ID NO: 19 (the amino terminal secretory signal of human IL-2; Met-Tyr-
Arg-Met-Gln-Leu-Leu-Ser-Cys-Ile-Ala-Leu-Ser-Leu-Ala-Leu-Val-Thr-Asn-Ser); SEQ ID
NO: 20 (cytokine signal sequence); Met-Tyr-Arg-Met-Ala-Leu-Leu-Ser-Cys-Ile-Ala-Leu-
Ser-Leu-Ala-Leu-Val-Thr-Asn-Ser); and SEQ ID NO: 21 (Met-Thr-Ser-Arg-Arg-Ser-Val-
Lys-Ser-Gly-Lys-Arg-Glu-Val-Lys-Arg-Asp-Glu-Tyr-Glu-Asp-Leu-Tyr-Tyr-Thr-Lys-Ser-
Ser-Gly-Ile-Ala-Ser-Lys-Asp-Ser-Lys-Lys-Asp-Thr-Ser-Arg-Arg-Gly-Ala-Leu-Gln-Thr-Arg
Ser-Arg-Gln-Arg-Gly-Glu-Val-Arg-Phe-Val-Gln-Tyr-Asp-Glu-Ser-Asp-Tyr-Ala-Leu-Tyr-
Gly-Gly-Ser-Ser-Ser-Glu-Asp-Asp-Glu-His-Pro-Glu-Val-Lys-Arg-Thr-Arg-Arg-Lys-Val-
Ser-Gly-Ala-Val-Leu-Ser-Gly-Lys-Gly-Lys-Ala-Arg-Ala-Lys-Lys-Lys-Lys-Ala-Gly-Ser-
Gly-Gly-Ala-Gly-Arg-Thr-Lys-Thr-Thr-Ala-Lys-Arg-Ala-Lys-Arg-Thr-Gln-Arg-Val-Ala-
Thr-Lys-Ala-Lys-Ala-Ala-Lys-Ala-Ala-Glu-Thr-Thr-Arg-Gly-Arg-Lys-Ser-Ala-Gln-Lys-
Glu-Ser-Ala-Ala-Leu-Lys-Asp-Ala-Lys-Ala-Ser-Thr-Ala-Lys-Thr-Arg-Ser-Lys-Thr-Lys-
Ala-Gln-Gly-Leu-Ala-Arg-Lys-Leu-His-Phe-Ser-Thr-Ala-Lys-Lys-Asn-Lys-Asp-Ala-Lys-
Trp-Thr-Lys-Arg-Val-Ala-Gly-Phe-Asn-Lys-Arg-Val-Phe-Cys-Ala-Ala-Val-Gly-Arg-Leu-
Ala-Ala-Met-His-Ala-Arg-Met-Ala-Ala-Val-Gln-Leu-Trp-Asp-Met-Ser-Arg-Lys-Arg-Thr-
Asp-Glu-Asp-Leu-Asn-Glu-Leu-Leu-Gly-Ile-Thr-Thr-Ile-Arg-Val-Thr-Val-Cys-Glu-Gly-
Lys-Asn-Leu-Leu-Gln-Arg-Ala-Asn-Glu-Leu-Val-Asn-Lys-Asp-Val-Val-Gln-Asp-Val-Asp-
Ala-Ala-Thr-Ala-Thr-Arg-Gly-Arg-Ser-Ala-Ala-Ser-Arg-Lys-Thr-Glu-Arg-Lys-Arg-Ala-
Lys-Ala-Arg-Ser-Ala-Ser-Arg-Lys-Arg-Arg-Lys-Val-Glu-Ser), SEQ ID NO:26 (IL-4 signal
sequence Met-Gly-Leu-Thr-Ser-Gln-Leu-Leu-Pro-Pro-Leu-Phe-Phe-Leu-Leu-Ala-Cys-Ala-
Gly-Asn-Phe-Val-His-Gly), SEQ ID NO:27 (VP22 Met-Thr-Ser-Arg-Arg-Ser-Val-Lys-Ser-
Gly-Pro-Arg-Glu-Val-Pro-Arg-Asp-Glu-Tyr-Glu-Asp-Leu-Tyr-Tyr-Thr-Pro-Ser-Ser-Gly-
Met-Ala-Ser-Pro-Asp-Ser-Pro-Pro-Asp-Thr-Ser-Arg-Arg-Gly-Ala-Leu-Gln-Thr-Arg-Ser-
Arg-Gln-Arg-Gly-Glu-Val-Arg-Phe-Val-Gln-Tyr-Asp-Glu-Ser-Asp-Tyr-Ala-Leu-Tyr-Gly-
Gly-Ser-Ser-Ser-Glu-Asp-Asp-Glu-His-Pro-Glu-Val-Pro-Arg-Thr-Arg-Arg-Pro-Val-Ser-
Gly-Ala-Val-Leu-Ser-Gly-Pro-Gly-Pro-Ala-Arg-Ala-Pro-Pro-Pro-Pro-Ala-Gly-Ser-Gly-
Gly-Ala-Gly-Arg-Thr-Pro-Thr-Thr-Ala-Pro-Arg-Ala-Pro-Arg-Thr-Gln-Arg-Val-Ala-Thr-
Lys-Ala-Pro-Ala-Ala-Pro-Ala-Ala-Glu-Thr-Thr-Arg-Gly-Arg-Lys-Ser-Ala-Gln-Pro-Glu-
Ser-Ala-Ala-Leu-Pro-Asp-Ala-Pro-Ala-Ser-Thr-Ala-Pro-Thr-Arg-Ser-Lys-Thr-Pro-Ala-
Gln-Gly-Leu-Ala-Arg-Lys-Leu-His-Phe-Ser-Thr-Ala-Pro-Pro-Asn-Pro-Asp-Ala-Pro-Trp-
Thr-Pro-Arg-Val-Ala-Gly-Phe-Asn-Lys-Arg-Val-Phe-Cys-Ala-Ala-Val-Gly-Arg-Leu-Ala-
Ala-Met-His-Ala-Arg-Met-Ala-Ala-Val-Gln-Leu-Trp-Asp-Met-Ser-Arg-Pro-Arg-Thr-Asp-
Glu-Asp-Leu-Asn-Glu-Leu-Leu-Gly-Ile-Thr-Thr-Ile-Arg-Val-Thr-Val-Cys-Glu-Gly-Lys-
Asn-Leu-Leu-Gln-Arg-Ala-Asn-Glu-Leu-Val-Asn-Pro-Asp-Val-Val-Gln-Asp-Val-Asp-Ala-
Ala-Thr-Ala-Thr-Arg-Gly-Arg-Ser-Ala-Ala-Ser-Arg-Pro-Thr-Glu-Arg-Pro-Arg-Ala-Pro-
Ala-Arg-Ser-Ala-Ser-Arg-Pro-Arg-Arg-Pro-Val-Glu-Gly), SEQ ID NO:28 (Arg-Arg-Arg-
Arg-Gly-Cys), SEQ ID NO:29 (Arg-Arg-Arg-Arg-Arg-Gly-Cys), SEQ ID NO:30 (Arg-Arg-
Arg-Arg-Arg-Arg-Gly-Cys), SEQ ID NO:31 (Arg-Arg-Arg-Arg-Arg-Arg-Arg-Gly-Cys),
SEQ ID NO:32 (Arg-Arg-Arg-Arg-Arg-Arg-Arg-Arg-Gly-Cys), SEQ ID NO:33 (Arg-Arg-
Arg-Arg-Arg-Arg-Arg-Arg-Arg-Gly-Cys), SEQ ID NO:34 (Arg-Arg-Arg-Arg-Arg-Arg-Arg
Arg-Arg-Arg-Gly-Cys), SEQ ID NO:35 (Arg-Arg-Arg-Arg-Arg-Arg-Arg-Arg-Arg-Arg-Arg-
Gly-Cys), SEQ ID NO:36 (Arg-Arg-Arg-Arg-Arg-Arg-Arg-Arg-Arg-Arg-Arg-Arg-Gly-Cys),
SEQ ID NO:37 (Arg-Arg-Arg-Arg-Arg-Arg-Arg-Arg-Arg-Arg-Arg-Arg-Arg-Gly-Cys), SEQ
ID NO:38 (Arg-Arg-Arg-Arg-Arg-Arg-Arg-Arg-Arg-Arg-Arg-Arg-Arg-Arg-Gly-Cys), SEQ
ID NO:39 (Arg-Arg-Arg-Arg-Arg-Arg-Arg-Arg-Arg-Arg-Arg-Arg-Arg-Arg-Arg-Gly-Cys),
SEQ ID NO:40 (Arg-Arg-Arg-Arg-Arg-Arg-Arg-Arg-Arg-Arg-Arg-Arg-Arg-Arg-Arg-Arg-
Gly-Cys), SEQ ID NO:41 (Arg-Arg-Arg-Arg-Arg-Arg-Arg-Arg-Arg-Arg-Arg-Arg-Arg-Arg-
Arg-Arg-Arg-Gly-Cys), SEQ ID NO:42 (Arg-Arg-Arg-Arg-Arg-Arg-Arg-Arg-Arg-Arg-Arg-
Arg-Arg-Arg-Arg-Arg-Arg-Arg-Gly-Cys), SEQ ID NO:43 (Arg-Arg-Arg-Arg-Arg-Arg-Arg-
Arg-Arg-Arg-Arg-Arg-Arg-Arg-Arg-Arg-Arg-Arg-Arg-Gly-Cys), SEQ ID NO:44 (Arg-Arg
Cys), and SEQ ID NO:45 (Kaposi's FGF signal sequence, full length Met, Ser, Gly, Asp,
Gly, Thr, Ala-Ala-Val-Ala-Leu-Leu-Pro-Ala-Val-Leu-Leu-Ala-Leu-Leu-Ala-Pro).

In certain preferred embodiments, the IRES is a viral IRES sequences, e.g., IRES sequences from picomaviriuses, flaviviruses, retroviruses, and herpesviruses as described in Vagner et al., EMBO reports 21(101):893-898 (2001) and Hellen and Sarnow, Genes & Gev. 15:1593-1612 (2001)). In a particularly preferred embodiment, the IRES is from encephalomyocarditis virus (e.g., nucleotides 1448-2030 of SEQ ID NO:46, nucleotides 5378-5936 of SEQ ID NO:46, or nucleotides 261-849 of GenBank Accession No. X73412). In other embodiments, the IRES sequences are mammalian IRES sequences (e.g., IRES sequences from c-myc, N-myc, c-jun, myt2, AML1/RUNX1, Gtx, Mnt,Nkx6.1, NRF, YAP1, Smad5, HIF-1 alpha, La autoantigen, eIF4GI, p97/DAPS/NAT1, XIAP, APC, Apaf-1, BAG-1, Bip/GRP78, FGF2, PDGF2/c-Sis, VEGF-A, IGF-II, Estrogen receptor alpha, IGF-1 receptor, Notch2, Connexin 43, Connexin 32, Cyr61, ARC, MAP2, Pim-1, p58 PITSLRE, alpha-CaM kinase II, CDK inhibitor p27, Protein kinase Cdelta, KV.14, Beta F1-ATPase, Cat-1, ODC, dendrin, Neurogranin/RC3, NBS1, FMR1, Rbm3, NDST (heparan sulfate/heparin GlcNAc N-deacetylase/N-sulfotransferase) as described in Vagner et al., supra 2001 and Hellen and Sarnow, supra 2001.

In certain embodiments, the eukaryotic promoter is a cytomegalovirus promoter, a vWf promoter, a CCSP/UG promoter, an osteoblast-specific osteocalcin promoter, an albumin promoter, a MCK promoter, a Muc-1 promoter, a CEA promoter, a PSA promoter, a HER-2 promoter, a Myc promoter, a L-plastin promoter, an AFP promoter, a HRE promoter, an egr-1 promoter, a mdr-1 promoter, a hsp70 promoter, a tetracycline induced promoter, a SV40 promoter, a ADH1 promoter, a GAL4 promoter, or a LexA promoter.

Suitable RNAP promoters include, for example, the following:

TAATACGACTCACTATAGGGAGA (SEQ ID NO: 22) for T7 RNAP,
ATTTAGGTGACACTATAGAAGAA (SEQ ID NO: 23) for SP6 RNAP,
AATTAACCCTCACTAAAGGGAGA (SEQ ID NO: 24) for T3 RNAP, and
AATTAGGGCACACTATAGGGAGA (SEQ ID NO: 25) for K11 RNAP.

Products of interest include, for example, a restriction endonuclease, a single-chain insulin, a cytokine, a non-therapeutic protein, a therapeutic protein. In certain embodiments, the product of interest is a therapeutic product. The therapeutic products can be chosen from a wide variety of compounds including, without limitation, a protein, a nucleic acid, an antisense nucleic acid, ribozymes, tRNA, snRNA, siRNA, and an antigen. In certain embodiments, the therapeutic product is a protein exemplified by proteins chosen from the following group: a herpes simplex virus thymidine kinase (HSV-TK), a cytosine deaminase, a xanthine-guaninephosphoribosyl transferase, a p53, purine nucleoside phosphorylase, and a cytochrome P450 2B1. In other embodiments, the therapeutic product is a protein selected from the group consisting of: p53, DAP kinase, p16, ARF, APC, neurofibromin, PTEN, WT1, NF1, an Apoptin, and VHL. In still other embodiments, the therapeutic product encodes a protein selected from the group consisting of: angiostatin, endostatin, and VEGF-R2. The therapeutic products can also be a cytokine, including without limitation: IL-2, IL-3, IL-4, IL-6, IL-7, IL-10, IL-12, IL-15, IFN-α, IFN-β, IFN-γ, TNF-α, GM-CSF, G-CSF, and Flt3-Ligand. Other therapeutic products include, without limitation, an antibody (e.g., a single chain antibody, a peptide hormone, EPO, a single-chain insulin, etc.

In yet another aspect, the present invention provides for compositions comprising a a vector comprising a secretable RNA polymerase expression cassette, wherein the expression cassette comprises (1) a eukaryotic promoter and a RNA polymerase promoter operably linked to a nucleic acid encoding a secretable RNA polymerase comprising a RNA polymerase, a secretion domain, and a first internal ribosome entry site; and (2) a RNA polymerase promoter operably linked to a nucleic acid encoding a product of interest and a second internal ribosome entry site, and a pharmaceutically acceptable carrier.

Another aspect of the invention provides for lipid-nucleic acid compositions comprising a nucleic acid-lipid particle comprising a lipid portion and a nucleic acid portion., the nucleic acid portion comprising a vector comprising a secretable RNA polymerase expression cassette as described herein. The sRNAP expression cassette comprises (1) a eukaryotic promoter and a RNA polymerase promoter operably linked to a nucleic acid encoding a secretable RNA polymerase comprising a RNA polymerase, a secretion domain, and a first internal ribosome entry site; and (2) a RNA polymerase promoter operably linked to a nucleic acid encoding a product of interest and a second internal ribosome entry site. In certain embodiments, the nucleic acid-lipid particle is a serum-stable nucleic acid-lipid particle comprising a nucleic acid fully encapsulated within the lipid portion. The lipid portion can be composed of a variety of different lipids and various proportions of lipids. In certain embodiments, the lipid portion contains a protonatable lipid having a pKa in the range of about 4 to about 11. In particularly preferred embodiments, the lipid portion contains a cationic lipid. Examples of cationic lipids include, without limitation, DODAC, DODAP, DODMA, DOTAP, DOTMA, DC-Chol, DMRIE, and DSDAC. In another preferred embodiment, the lipid portion contains a bilayer stabilizing component, such as a PEG-lipid derivative (e.g., a PEG diacylglycerol as described in U.S. patent application Ser. No. 09/895,480, or a PEG-dialkyloxypropyl as described in U.S. patent application Ser. No, 60/503,239, filed Sep. 15, 2003 (Attorney Docket No. 020801-002000US)) or an ATTA-lipid derivative

In yet another aspect, the present invention provides methods of expressing a nucleic acid encoding a product of interest in a cell. These methods involve introducing into a cell an expression cassette comprised of a RNA polymerase promoter operably linked to a nucleic acid encoding a product of interest; and contacting the cell with a secretable RNA polymerase comprising a RNA polymerase and a secretion domain. In certain embodiments, the cell contains a secretable RNA polymerase expression cassette comprised of a eukaryotic promoter operably linked to a nucleic acid encoding a secretable RNA polymerase, wherein the secretable RNA polymerase contains a RNA polymerase and a secretion domain. In certain embodiments, the secretable RNA polymerase is expressed from a cell comprising a secretable RNA polymerase expression cassette comprised of a eukaryotic promoter operably linked to a nucleic acid encoding a secretable RNA polymerase, wherein the secretable RNA polymerase contains a RNA polymerase and a secretion domain. In other embodiments, the secretable RNA polymerase being contacted with the cell is a purified secretable RNA polymerase. Preferably the expression cassette encoding the therapeutic product is present on the same nucleic acid molecule as the secretable RNA polymerase expression cassette.

In still yet another aspect, the present provides for methods of treating a disease in a subject, involving administering a therapeutically effective amount of an expression cassette comprised of a RNA polymerase promoter operably linked to a nucleic acid encoding a therapeutic product, and administering a therapeutically effective amount of a secretable RNA polymerase, wherein the secretable RNA polymerase comprises a RNA polymerase and a secretion domain. In certain embodiments, the secretable RNA polymerase is expressed from a secretable RNA polymerase expression cassette comprising a eukaryotic promoter operably linked to a nucleic acid encoding a secretable RNA polymerase, wherein the secretable RNA polymerase contains a RNA polymerase and a secretion domain. In certain embodiments, the secretable RNA polymerase expression cassette further contains a RNA polymerase promoter operably linked to the nucleic acid encoding a secretable RNA polymerase. Preferably the expression cassette encoding the therapeutic product is present on the same nucleic acid molecule as the secretable RNA polymerase expression cassette. In other embodiments, the expression cassette encoding the therapeutic product is present on a first nucleic acid molecule and the secretable RNA polymerase expression cassette is present on a second nucleic acid molecule.

The therapeutic products used in these methods can essentially be any therapeutic product that is efficacious in the treatment, amelioration, or prevention of a disease or condition. Examples of diseases and conditions that can be treated using the methods of the present invention include, without limitation, the following: cancer, autoimmune disease, hemophilia, arthritis, cardiovascular disease, cystic fibrosis, sickle cell anemia, infectious disease, viral disease, AIDS, herpes, bacterial disease, pneumonia, tuberculosis and an inflammatory disease. Examples of therapeutic products include, without limitation, a protein, a nucleic acid, an antisense nucleic acid, and an antigen. In certain embodiments, enzymes and proteins that are cytotoxic by themselves or in conjunction with a prodrug are useful in treating cancer and other conditions. These enzymes and proteins include, without limitation, a herpes simplex virus thymidine kinase (HSV-TK), a cytosine deaminase, a xanthine-guaninephosphoribosyl transferase, a p53, a purine nucleoside phosphorylase, a carboxylesterase, a deoxycytidine kinase, a nitroreductase, a thymidine phosphorylase, and a cytochrome P450 2B1. In other embodiments, cytokines and immunomodulators are useful as therapeutic products when used in methods of the present invention. Examples of useful cytokines include, without limitation, the following: IL-2, IL-3, IL-4, IL-6, IL-7, IL-10, IL-12, IL-15, IFN-α, IFN-β, IFN-γ, TNF-α, GM-CSF, G-CSF, and Flt3-Ligand.

These and other aspects of the present invention will become apparent upon reference to the following detailed description.

### BRIEF DESCRIPTION OF THE DRAWINGS

FIG. 1 depicts a secretable RNA polymerase expression cassette of the present invention.

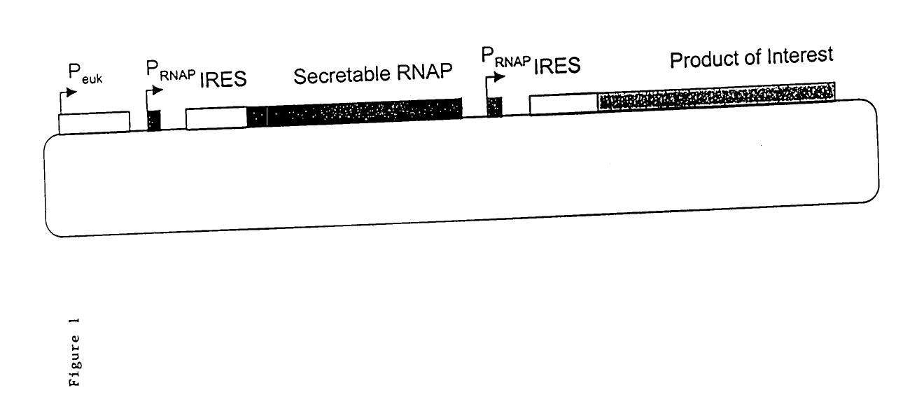

FIG. 2 illustrates in vitro transfection of Neuro 2A cells with Tat-RNAP (Tat: SEQ ID NO:1). Neuro 2A cells were transfected with T7-luciferase and CMV-Tat-RNAP constructs in DOPE:DODAC (50:50) large unilamellar vesicles (LUVs).Cells were harvested 24, 48, and 72 hours after transfection and luciferase activity was measured.

FIG. 3 illustrates in vitro transfection of BHK cells with VP22-RNAP (VP22: SEQ ID NO:21). BHK cells were transfected with T7-luciferase and CMV-VP22-RNAP constructs in DOPE:DODAC (50:50) large unilamellar vesicles (LUVs). Cells were harvested 24, 48, and 72 hours after transfection and luciferase activity was measured.

FIG. 4 illustrates in vitro transcription and translation of VP22-RNAP. 500 ng of a SP6-VP22-T7-RNAP (VP22: SEQ ID NO:21) construct was added to 250 ng of a T7-luciferase construct and 1 μl of SP6 RNA polymerase. Luciferase activity was measured over time.

FIG. 5 illustrates in vitro transcription and translation of Tat-RNAP. 500 ng of a SP6-Tat-T7-RNAP (Tat: SEQ ID NO:1) construct was added to 250 ng of a T7-luciferase construct and 1 μl of SP6 RNA polymerase. Luciferase activity was measured over time.

FIG. 6 illustrates in vitro transfection and translation of Tat-RNAP and luciferase. BHK cells were transfected with 5, 50, or 250 nmol of purified Tat-RNAP (Tat: SEQ ID NO: 1) for 4 hours, washed with PBS, and transfected with 0.75 μg of a T7-luciferase construct.

FIG. 7 illustrates in vitro transfection of VP22-RNAP. BHK cells were transfected with 1 μg of a CMV-T7 RNAP construct or a CMV-VP22-T7RNAP construct (VP22: SEQ ID NO:21). Four hours after transfection, the BHK cells were trypsinized and added to BHK cells transfected with T7-luciferase. Cells were harvested 24, 48, or 72 hours after mixing of the cell populations and luciferase activity was measured.

FIG. 8 depicts plasmid diagrams of major constructs used. R023 is an autogene construct, containing the T7 RNAP gene driven by the T7, T3 and SP6 promoters (PTRI). L059 is the luciferase reporter gene cassette. R011 is a bi-cistronic autogene construct (R023+L059). L053 is the CMV driven nuclear expression construct.

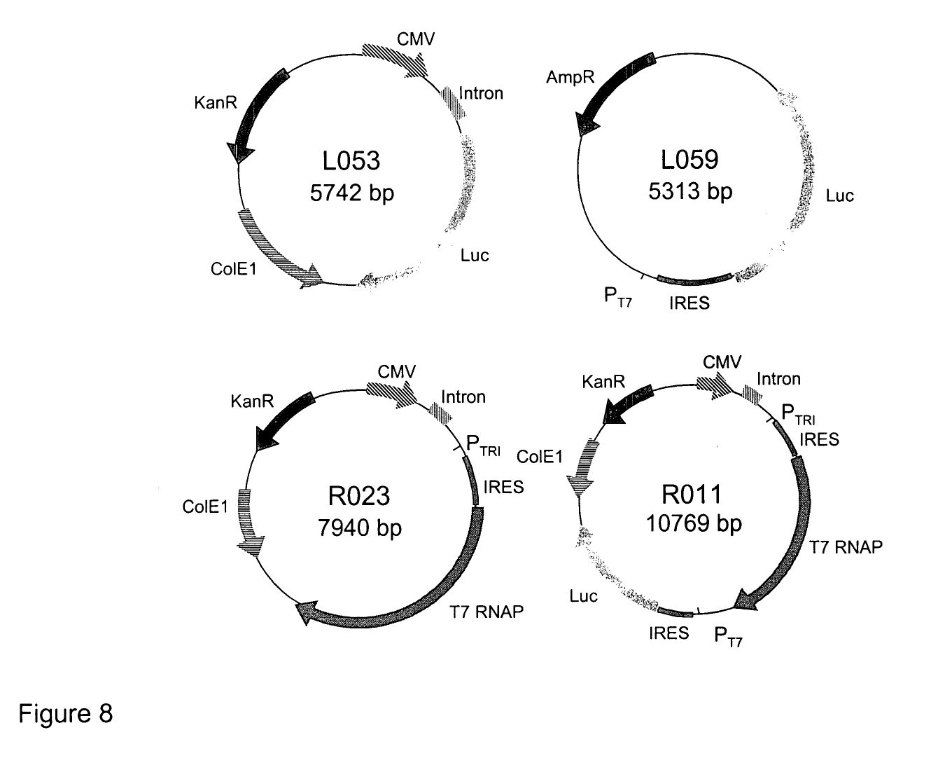

FIG. 9 describes transcription and translation assays: FIG. 9A is a schematic diagram of the transcription and translation assay. SP6 RNAP binds to the SP6 promoter (PSP6) on R023 (T7 RNAP driven by SP6 and T7 promoters) (1) transcribing T7 RNAP mRNA, which is (2) translated into T7 RNAP protein. The T7 RNAP protein then binds the T7 promoter (PT7) on R023 (3) resulting in more T7 RNAP protein (2) and initiating the autocatalytic cycle and an exponential increase in T7 RNAP production. T7 RNAP also transcribes luciferase mRNA from PT7-Luc (4), resulting in an increase in luciferase expression proportional to the amount of T7 RNAP present. In the control reaction (below), the lack of PT7 in R037 (T7 RNAP gene driven by only SP6 promoter) prevents any autocatalytic production of T7 RNAP (3). FIG. 9B illustrates data from an in vitro coupled transcription and translation (Promega) assay. 250 ng of PT7-Luc was combined with 250 ng of either R023 or R037 in a total reaction volume of 15 μl and 0.5 U of SP6 RNAP (Promega) was added and incubated at 30° C. 2 μl aliquots were removed at time points indicated and subjected to luciferase analysis as described in Materials and Methods. After an initial lag phase, the R023 reaction resulted in an exponential increase in luciferase expression, verifying the autocatalytic nature of the system.

FIG. 10 illustrates the comparison of bi-cistronic construct versus a dual plasmid transfection. BHK cells were transfected with 1 μg/well of plasmid. Equimolar amounts of plasmids were added, and the total mass of DNA per transfection was kept equal by adding an unrelated plasmids (pBlueScript). Transfections and luciferase assays were performed as described in Materials and Methods. Error bars indicate standard error. Transfection with the bi-cistronic autogene construct (R011) resulted in expression levels that were two to four-fold higher than the dual plasmid transfection (autogene and reporter gene on separate plasmids). There is no luciferase expression in the absence of the autogene cassette.

FIG. 11 illustrates data demonstrating that plasmid size does not effect transfection of BHK cells. BHK cells were transfected with a total of 1 μg/well. Equimolar amounts of plasmid were added, and the total mass of DNA per transfection was normalized by adding an unrelated plasmid (pBlueScript). Error bars indicate standard error. The size of plasmid, ranging from 5.8 kb to 10.8 kb does not have an effect on transfection in BHK cells.

FIG. 12 illustrates data comparing nucleic acid expression in cells transfected with an autogene construct and cells transfected with a standard nuclear expression plasmid. BHK cells were transfected with a total of 1 μg/well. Equimolar amounts of plasmids were added, and the total mass of DNA per transfection was kept equal by adding an unrelated plasmid (pBlueScript). Error bars indicate standard error. Transfection with the autogene (R011) yielded a 20-fold increase in expression over the standard nuclear expression plasmid (L053).

FIG. 13 is a graphic illustration of a primer extension assay performed on BHK cells transfected with the bi-cistronic autogene construct (R011). The transcripts initiated at the nuclear CMV promoter are predicted to have a longer 5′ untranslated region resulting in larger fragments, ˜300 bp in size, while transcripts initiated at the T7 promoter are predicted to have a shorter 5′ untranslated region, ˜90 bp in size.

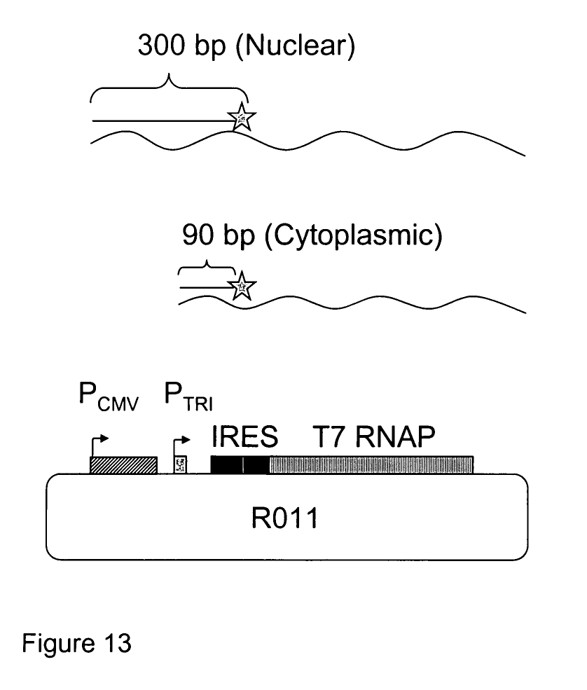

FIG. 14 illustrates data showing that increased autogene expression is also seen in Neuro2A cells. Neuro2A cells were transfected with a total of 2 μg/well. Equimolar amounts of plasmids were added, and the total mass of DNA per transfection was kept equal by adding an unrelated plasmid (pBlueScript). Error bars indicate standard error. Transfection with the autogene (R011) yielded a 20-fold increase in expression over the standard nuclear expression plasmid (L053). data from a Ribonuclease Protection Assay of RNA derived from BHK cells transfected with bi-cistronic autogene construct (R011) or nuclear construct (L053). BHK cells were treated with Actinomycin D 24 h post transfection. Total RNA was harvested at 2-h intervals following treatment. 10, 5 or 2.5 μg of total RNA was subjected to an RNase Protection Assay using 32P labeled probes against T7 RNAP (RNAP) and Luciferase (Luc) transcripts. All values were standardized against the GAPDH control. Approximately 20 times as many luciferase transcripts were detected in the autogene transfected cells as the nuclear transfected cells. The half-life of the autogene transcripts is approx 103 min, approximately 3-fold shorter than the half-life of the nuclear transcripts, 317 min.

### DETAILED DESCRIPTION OF THE INVENTION

I. Introduction

The present invention provides nucleic acids and methods of expressing a product of interest in a cell. In some embodiments, the nucleic acids are vectors (i.e., bicistronic autogene constructs) comprising expression cassettes comprising (1) a eukaryotic promoter and a first RNA polymerase promoter operably linked to a nucleic acid encoding a secretable RNA polymerase comprising a RNA polymerase and a secretion domain, and a first internal ribosome entry site (IRES); and (2) a second RNA polymerase promoter operably linked to a nucleic acid encoding a product of interest and a second IRES. To express a product of interest, the expression cassette is introduced into a suitable cell. Typically the expression cassette encoding the therapeutic product is present on the same nucleic acid molecule as the

In other embodiments, the invention involves generating sRNAPs that are then contacted with and enter a cell that contains an expression cassette with a RNAP promoter operably linked to a nucleic acid encoding a product of interest. Preferably the expression cassette encoding the therapeutic product is present on the same nucleic acid molecule as the secretable RNA polymerase expression cassette.

In both of the embodiments described above, the product of interest can be a product that is purified and used as a pharmaceutical (e.g., single-chain insulin, EPO, a cytokine, etc.). In other embodiments, the products of interest are therapeutic products that are expressed in a subject suffering from a disease. The production of a therapeutically effective amount of the therapeutic product in the subject is useful for the treatment of the disease that is afflicting the subject. These methods and components will be described in more detail below.

II. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “RNA polymerase” (RNAP) refers to a protein that is able to catalyze the polymerization of RNA from DNA.

An “Internal Ribosome Entry Site” or “IRES” efers to a nucleic acid motif which forms a structure that allows proper alignment of ribosome subunits and other co-factors for translation of mRNA. Suitable IRES include, for example, Viral Internal Ribosome Entry Sites (IRES), such as the EMCV (encephalopmyocarditis virus), FMDV (Foot and mouth disease), and other picornaviruses based IRES sequences (see, e.g., Agol, Adv. Virus Res. 40: 103-80 (1991); Jackson, et al. Mol. Biol. Rep. 19(3): 147-59 (1994); and Jackson and Kaminski (1995). RNA 1(10): 985-1000 (1995)). Typically the structure is one that can conveniently be used to initiate cap-independent mRNA translation, which is component of the autogene based cytoplasmic expression system.

A “secretable RNA polymerase” is a molecule that contains a RNA polymerase linked to a secretion domain. A “secretable RNA Polymerase” (sRNAP) is able to enter the cytoplasm of a cell when contacted with the cell.

A “secretion domain” is a polypeptide sequence that when linked to another polypeptide creates a fusion protein that is able to enter a cell when contacted with that cell. Examples of secretion domains include, without limitation, SEQ ID NOS: 1, 2, 3, 4, 5, 6, 7, 8, 9, 10, 11, 12, 13, 14, 15, 16, 17, 18, 19, 20, and 21.

A “non-host RNA polymerase” is a RNAP that is not naturally encoded by the nuclear genome of a eukaryotic organism.

A “phagemid RNA polymerase” is a RNAP from a bacteriophage (e.g., T3, T7, SP6, and K11 bacteriophages).

A “SP6 RNA Polymerase” is a RNAP encoded by a nucleic acid that is about 90% or more identical to GenBank Accession No. Y00105 or a nucleic acid that hybridizes under stringent conditions to the complement of the nucleic acid set forth in GenBank Accession No. Y00105.

A “T7 RNA Polymerase” is a RNAP encoded by a nucleic acid that is about 90% or more identical to GenBank Accession No. M38308 or a nucleic acid that hybridizes under stringent conditions to the complement of the nucleic acid set forth in GenBank Accession No. M38308.

A “K11 RNA Polymerase” is a RNAP encoded by a nucleic acid that is about 90% or more identical to GenBank Accession No. X53238 or a nucleic acid that hybridizes under stringent conditions to the complement of the nucleic acid set forth in GenBank Accession No. X53238.

A “T3 RNA Polymerase” is a RNAP encoded by a nucleic acid that is about 90% or more identical to GenBank Accession No. X02981 or a nucleic acid that hybridizes under stringent conditions to the complement of the nucleic acid set forth in GenBank Accession No. X53238.

One of skill in the art will appreciate that stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

For the purpose of the invention, suitable “moderately stringent conditions” include, for example, prewashing in a solution of 5×SSC, 0.5% SDS, 1.0 mM EDTA (pH 8.0), hybridizing at 50° C.-65° C., 5×SSC overnight, followed by washing twice at 65° C. for 20 minutes with each of 2×, 0.5× and 0.2×SSC (containing 0.1% SDS).

An “expression cassette” is a polynucleotide sequence that contains a nucleic acid coding sequence for a protein, polypeptide, antisense nucleic acid, sense nucleic acid, etc., and the necessary control elements (e.g., promoter sequence(s), transcription start site, translation start site, etc) for expression of the nucleic acid coding sequence. One or more expression cassettes can be on a single nucleic acid molecule, e.g,. a plasmid, a vector, etc.

A “secretable RNA polymerase expression cassette” is an expression cassette that encodes a secretable RNA polymerase (sRNAP).

The term “eukaryotic promoter” refers to a nucleic acid sequence that when operably linked to a nucleic acid, permits transcription of that nucleic acid in the nucleus of a eukaryotic cell.

A promoter is “operably linked” to a nucleic acid when the relationship between the promoter and the nucleic acid is such that expression of the nucleic acid can take place. The promoter does not have to be contiguous with the nucleic acid, i.e., there can be intervening nucleic acid sequences between the nucleic acid and the promoter. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence. DNA regions are “operably linked” when they are functionally related to each other. For example, DNA for a signal peptide (secretory leader) is operably linked to DNA for a polypeptide if it is expressed as a precursor which participates in the secretion of the polypeptide; a promoter is “operably linked” to a coding sequence if it controls the transcription of the sequence; or a ribosome binding site is “operably linked” to a coding sequence if it is positioned so as to permit translation. Generally, “operably linked” means contiguous and, in the case of secretory leaders, in reading frame. DNA sequences encoding immunogenic polypeptides which are to be expressed in a microorganism will preferably contain no introns that could prematurely terminate transcription of DNA into mRNA.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; Rossolini et al. (1994) Mol. Cell. Probes 8:91-98). The term nucleic acid is used interchangeably with gene, cDNA, mRNA, oligonucleotide, and polynucleotide.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 90%, 95%, or 99% or higher, compared to a reference sequence using the programs described herein, preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. “Substantial identity” of amino acid sequences for these purposes normally means that a polypeptide comprises a sequence that has at least 40% sequence identity to the reference sequence. Preferred percent identity of polypeptides can be any integer from 40% to 100%. More preferred embodiments include at least 60%, 65%, 70%, 75%, 80%, 85%, 90%, 95%, or 99%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

Optimal alignment of sequences for comparison may be conducted by the local identity algorithm of Smith and Waterman (1981) Add. APL. Math. 2:482, by the identity alignment algorithm of Needleman and Wunsch (1970) J. Mol. Biol. 48:443, by the search for similarity method of Pearson and Lipman (1988) Proc. Natl. Acad. Sci. U.S.A. 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

A preferred example of algorithms that are suitable for determining percent sequence identity and sequence similarity are the BLAST and BLAST 2.0 algorithms, which are described in Altschul et al. (1977) Nuc. Acids Res. 25:3389-3402 and Altschul et al. (1990) J. Mol. Biol. 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

A “RNA polymerase promoter” is a nucleic acid comprising a sequence of nucleotides to which a RNA polymerase can bind to and activate transcription.

A “therapeutic product” is a compound, (e.g., a protein, a nucleic acid, a hormone, an antisense nucleic acid, an antigen, etc.) that can be used to treat or ameliorate a disease or condition.

The term “serum-stable” in relation to a nucleic acid-lipid particle means that the nucleic acid is fully encapsulated by the lipid portion of the nucleic acid-lipid particle such that less than 5% of the nucleic acid is degraded after exposure of the nucleic acid-lipid particle to 1 U DNAse I for 30 minutes in digestion buffer at 37° C.

The term “cationic lipid” refers to any of a number of lipid species that carry a net positive charge at a selected pH, such as physiological pH. Such lipids include, but are not limited to, N,N-dioleyl-N,N-dimethylammonium chloride (“DODAC”); N-(2,3-dioleyloxy)propyl)-N,N,N-trimethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3-(N-(N′,N′-dimethylaminoethane)-carbamoyl)cholesterol (“DC-Chol”) and N-(1,2-dimyristyloxyprop-3-yl)-N,N-dimethyl-N-hydroxyethyl ammonium bromide (“DMRIE”). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (“DOSPA”) and(“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA and the like.

A “purified secretable RNA polymerase” is a secretable RNAP that is at least 50% pure.

“Therapeutically effective amount,” as used herein, refers to an amount of a compound (e.g., drug, nucleic acid, etc.) that is sufficient or necessary to give rise to a desired therapeutic effect. The therapeutic effect can be obtained directly or indirectly. For instance, the therapeutic agent can lead to activation of other therapeutic agents or can act in combination with additional therapeutic agents. For neoplasia, a therapeutic effect can be, for example, a reduction in growth, inhibition or reduction in size of the neoplasia or inhibition or reduction of metastasis and other malignant attributes, or other beneficial effects, such as subjective or objective observations of physicians and patients.

III. Secretable RNAPs

The secretable RNAPs of the present invention comprise a secretion domain and a RNAP domain. Typically, RNAPs are not secretable in that they are not secreted from cells and are not able to enter a cell. However, there are protein sequences known in the art, secretion domains, that when attached to a cargo peptide, that is not secretable, generates a secretion domain fused to a cargo peptide that is competent to enter a cell. Thus, the attachment of a secretion domain to the N— or C-terminus of a RNAP generates a secretable RNAP (sRNAP). The secretion domain may be expressed as a fusion protein comprising the secretion domain and the RNAP domain or can be the result of chemically linking the secretion domain to the RNAP domain. The connection between the secretion domain and the fusion protein can be direct or there can be a linker between them. The presence of a linker can be advantageous for the function of the molecule.

A. RNA Polymerases

It is preferred that the RNAP is a non-host RNA Polymerase that is active in the cytoplasm of a eukaryotic cell. Examples of RNAPs that are useful in the present invention include, without limitation, a phagemid RNA polymerase, a prokaryotic RNA polymerase, an archaebacterial RNA polymerase, a plant RNA polymerase, a fungal RNA polymerase, a eukaryotic RNA polymerase, a viral RNA polymerase, a mitochondrial RNA polymerase, and a chloroplast RNA polymerase. In particularly preferred embodiments, the phagemid RNAP is from a bacteriophage and encodes a single chain RNAP that is active as a monomer or higher order homomer (e.g., dimer). Particularly preferred phagemid RNAPs include, a SP6 RNAP (e.g., GenBank Accession No. Y00105), a T7 RNAP (e.g., GenBank Accession No. M38308), a T3 RNAP (e.g., GenBank Accession No X02981), and a K11 RNAP (e.g., GenBank Accession No. X53238; (Dietz et al. (1990) Mol. Gen. Genet. 221: 283-286). These phagemid RNAPs have been cloned and expressed in bacteria and several are commercially available (e.g,. SP6 RNAP, T7 RNAP, T3 RNAP). For example, the T7 RNAP (Davanloo et al. (1984) Proc. Natl. Acad. Sci., U.S.A. 81: 2035-2039 ) and the K11 RNAP (Han et al. (1999) Protein Expr. Purif. 16: 103-108) have been expressed as soluble proteins in E. coli.

The sRNAPs of the present invention should retain the enzymatic activity of the native RNAP, i.e., the ability to carry out template dependent synthesis of RNA. For example, the functionality of a sRNAP can be assessed using in vitro transcription and translation assays. One such assay utilizes a commercially available rabbit reticulocyte lysate, a cell-free reagent which contains all of the ribosomes and components needed for transcription and translation. The cell-free lysate is incubated with the sRNAP and a plasmid encoding a luciferase reporter plasmid. The luciferase reporter plasmid has a RNAP promoter specific for the sRNAP operably linked to a luciferase gene. If the sRNAP is able to transcribe the luciferase gene, then luciferase will be present in the sample and can be assayed using a luminometer.

In addition, the sRNAPs should be able to enter into a cell. One method of assaying whether a sRNAP can enter a cell is to tranfect two separate populations of cells. The first population is transfected with a nucleic acid comprising a sRNAP expression cassette. The second population of cells is tranfected with a nucleic acid comprising a luciferase reporter plasmid that has a RNAP promoter specific for the sRNAP operably linked to a luciferase gene or a product of interest. After the transfection, the two populations are mixed and luciferase activity is assayed. The presence of luciferase will confirm that the sRNAP protein was transported inter-cellularly in order to activate luciferase expression in neighboring cells. Similarly, an assay for the product of interest can be carried out to test whether the sRNAP is functional. Alternatively, purified sRNAP or cell culture media from the first population of cells just described is incubated with the second population of cells comprising the RNAP promoter driven luciferase expression cassette. The presence of luciferase activity is an indication that the sRNAP can enter into a cell.

B. Secretion Domain

The secretion domains when fused to the RNAP should generate a sRNAP. That is the sRNAP will have the ability to enter a cell from the outside and pass into the cytoplasm, such that the sRNAP can carry out transcription of an expression cassette containing a RNAP promoter. In certain embodiments of the present invention, the secretion domain targets the sRNAP to the cytoplasm of the cell. For example, the secretion domains can be chosen from the following secretion domains: SEQ ID NOS: 1, 2, 3, 4, 5, 6, 7, 8, 9, 10, 11, 12, 13, 14, 15, 16, 17, 18, 19, 20, 21, 26, 27, 28, 29, 30, 31, 32, 33, 34, 35, 36, 37, 38, 39, 40, 41, 42, 43, 44, and 45.

Several classes of secretion domains are known in the art. Examples of classes of secretion domains include signal peptides and protein transduction domains, both of which are described below.

1. Signal Peptides

Signal peptide sequences are hydrophobic peptides that mediate translocation of many secretory proteins across membranes (see, von Heijne (1990) J. Membrane Biol. 115: 195-201). Signal peptide sequences can be chosen from databases, such as the SIGPEP database (von Heijne (1987) Protein Sequence Data Analysis 1: 41-42; von Heijne and Abrahmsen (1989) FEBS Letters 224: 439-446). Examples of signal peptides include the signal peptide sequences for IL-2 (e.g., SEQ ID NOS: 19 and 20).

A particularly preferred class of signal peptides that can be used as secretion domains are importation competent signal peptides which permit cargo peptides to be imported into a cell as an importation competent signal peptide-cargo fusion protein (see, e.g., U.S. Pat. No. 5,807,746 and U.S. Pat. No. 6,043,339). An importation competent signal peptide is hydrophobic in nature and comprises about 55-60% hydrophobic residues such that it is capable of being secreted from a cell and can penetrate a cell membrane when contacted with the outside of the cell. In certain embodiments, the importation competent signal peptide is a sequence of amino acids generally of a length from about 10 to about 50 or more amino acids. A preferred importation competent signal peptide is SEQ ID NO: 18, the signal peptide of K-FGF (Kaposi Fibroblast growth factor).

2. Protein Transduction Domains

Protein transduction domains (PTDs) have been described in the art and are small regions of proteins that have the ability to traverse biological membranes in a receptor and transporter-independent manner (reviewed in Schwarze and Dowdy (2000) Trends Pharmacol. Sci. 21(2):45-48). Cargo proteins when linked to protein transduction domains can also traverse biological membranes (see, Schwarze and Dowdy (2000) Trends Pharmacol. Sci. 21(2):45-48). Examples of PTDs include, without limitation, VP22, Tat, and the third helix of the Drosophila homeodomain transcription factor ANTP. The minimal regions for these PTDs have been described as being residues 47-57 of Tat, residues 267-300 of VP22, and residues 43-58 of ANTP.

a) VP22 Peptides and VP22 Analog Peptides

A Herpesvirus structural protein, VP22, when fused to cargo proteins can be rapidly taken up by eukaryotic cells (see, e.g., U.S. Pat. No. 6,017,735; U.S. Pat. No. 6,184,038; Elliott and O'Hare (1997) Cell 88(2):223-233; Elliott and O'Hare (1999) Gene Ther. 6(1):149-151; and Aints et al. (1999) J. Gene Med. 1:275-279). This uptake process appears to occur via a non-classical Golgi-independent mechanism. VP22 can be fused to the N— or C-terminus of a heterologous protein to generate a secretable protein. In addition, VP22-fusion protein import and export does not appear to be limited to particular cell type (Elliott and O'Hare (1997); Wybranietz et al. (1999) J. Gene Med. 1(4):265-274). For example, VP22-GFP proteins were expressed by and spread intercellularly by cell types such as HepG2 (human hepatoma), Hep3B (human hepatoma), HuH7 (human hepatoma), HeLa (human cervix adenocarcinoma), MCF-7 (human mammary carcinoma), HEK-293 (human embryo kidney), CV-1 (monkey kidney), COS-1 (monkey kidney), NIH-3T3 (mouse fibroblast), and M-12 (canine kidney) (Wybranietz et al. (1999) J. Gene Med. 1(4):265-274)). A VP22-p53 and a p53-VP22 fusion protein were both able to efficiently induce apoptosis in p53 negative osteosarcoma cells, indicating that these proteins are useful for inducing cytotoxicity in tumorigenic cells (Phelan et al. (1998) Nat. Biotechnol. 16(5):440-443). Similarly, VP22-tk and tk-VP22 fusion proteins were effective at killing cells in vitro and a neuroblastoma tumor in vivo when ganciclovir was co-administered (Dilber et al. (1999) Gene Ther. 6(1):12-21).

b) Antennapedia Third Helix Peptides

Peptides comprising the third Helix of the ANTP transcription factor (e.g., amino acids 43-58) when fused to a cargo oligopeptide or cargo oligonucleotides can be translocated across a plasma membrane (Derossi et al. (1998) Trends Cell Biol. 8:84-87). For example, U.S. Pat. No. 5,888,762 describes macromolecules that are able to enter a living cell by virtue of a peptide fragment corresponding to the third helix of the Antennapedia homoeodomain (residues 43-58). Examples of useful Antennapedia third helix sequences are SEQ ID NOS: 10, 11, 12, 13, 14, 15, 16, and 17 (Prochiantz (2000) Curr. Opin. Cell Biol. 12:400-406; Derossi et al. (1998)).

c) TAT Peptides and Analogs Thereof

In certain embodiments of the present invention, the protein transduction domain is comprised of a tat sequence or a variant thereof. Tat sequences, and variant thereof, have been heterologously fused to cargo peptides. These tat-cargo peptides are able to enter cells by contacting them with the outside of the cell. Tat sequences that are useful as secretion domains include, without limitation, SEQ ID NOS: 1, 2, 3, 4, 5, 6, 7, and 8 (see, e.g., WO 99/29721; WO 00/34308 and WO 00/62067). For example, when a 11-amino acid protein transduction domain from the HIV TAT protein was fused to β-galactosidase, a cell permeable Tat-βgal protein was created (see, Schwarze et al. (1999) Science 285(5433):1569-1572). When the Tat-β-gal protein was injected ip into a mouse, staining for β-gal activity was found throughout the animal, including the heart, liver, kidney, lung, and muscle. Staining was also found in the brain, indicating that the tat-fusion proteins have the ability to cross the blood-brain barrier.

Methods for generating transducible Tat fusion proteins are known in the art (see e.g., Vocero-Akbani et al. (2000) Methods Enzymol. 322:508-521). The Tat fusion proteins can be tagged with an oligohistidine stretch on the N-terminus to facilitate purification. (Vocero-Akbani et al. (2000)). For example, a histidine tagged Tat domain (Arg-Lys-Lys-Arg-Arg-Gln-Arg-Arg-Arg) when fused to the N-terminus of superoxide dismutatase (SOD) generates a Tat-SOD that can be expressed in E. coli and can enter HeLa cells when added to culture media (Kwon et al. (2000) FEBS Lett. 485(2-3):163-167).

One of skill in the art can screen sRNAPs to see if a particular secretion domain confers the ability to enter cells using a variety of methods known to those of skill in the art. For example, the sRNAPs (or other secretion domain fusion proteins) can be labeled with a detectable label, such as a fluorescent label (e.g., fluorescein), and followed by FACS analysis (Vocero-Akbani et al. (2000)). In certain embodiments, purified denatured secretion domain fusion proteins are employed which can increase the efficiency of the biological response being measured or effected (see, e.g., Vocero-Akbani et al. (2000)).

3. Linker Regions

The secretion domains can be directly fused to the RNAP or a linker region (e.g., of amino acids) can be used to join the secretion domain to the RNAP. If the linker region is comprised of amino acids, then the linker sequence is preferably between 1 and 2-30 amino acids. The composition and arrangement of the amino acids in the linker region should permit the RNAP to retain its activity and allow the sRNAP to enter a cell.

C. Expression Cassettes Encoding a sRNAP

One way to generate the sRNAPs used in the present invention is to express them in a eukaryotic cell. In preferred embodiments, the sRNAPs are expressed from a cell containing an expression vector comprising a secretable RNA polymerase expression cassette. The expression cassette typically comprises two components: (a) a eukaryotic promoter, a first RNA polymerase promoter operably linked to a nucleic acid encoding a secretable RNA polymerase having a secretion domain, and a first internal ribosome entry site (IRES); and (b) a second RNA polymerase promoter operably linked to a nucleic acid encoding a product of interest (i.e., a heterologous nucleic acid) and a second internal ribosome entry site.

1. IRES

An “Internal Ribosome Entry Site” or “IRES” can conveniently be used to initiate translation of both the secretable RNA Polymerase and the product of interest.

One of skill in the art will appreciate that any IRES can be used in the expression cassettes described herein. Suitable IRES include, for example, Viral Internal Ribosome Entry Sites (IRES), such as the EMCV (encephalopmyocarditis virus), FMDV (Foot and mouth disease), and other picornaviruses based IRES sequences (see, e.g., Agol, Adv. Virus Res. 40: 103-80 (1991); Jackson, et al. Mol. Biol. Rep. 19(3): 147-59 (1994); and Jackson and Kaminski (1995). RNA 1(10): 985-1000 (1995)). In certain preferred embodiments, the IRES is a viral IRES sequences, e.g., IRES sequences from picornaviriuses, flaviviruses, retroviruses, and herpesviruses as described in Vagner et al., EMBO reports 21(101):893-898 (2001) and Hellen and Sarnow, Genes & Gev. 15:1593-1612 (2001)). In a particularly preferred embodiment, the IRES is from encephalomyocarditis virus (e.g., nucleotides 1448-2030 of SEQ ID NO:46, nucleotides 5378-5936 of SEQ ID NO:46, or nucleotides 261-849 of Genbank Accession No. X73412). In other embodiments, the IRES sequences are mammalian IRES sequences (e.g., IRES sequences from c-myc, N-myc, c-jun, myt2, AML1/RUNX1, Gtx, Mnt,Nkx6.1, NRF, YAP1, Smad5, HIF-1 alpha, La autoantigen, eIF4GI, p97/DAP5/NAT1, XIAP, APC, Apaf-1, BAG-1, Bip/GRP78, FGF2, PDGF2/c-Sis, VEGF-A, IGF-II, Estrogen receptor alpha, IGF-1 receptor, Notch2, Connexin 43, Connexin 32, Cyr61, ARC, MAP2, Pim-1, p58 PITSLRE, alpha-CaM kinase II, CDK inhibitor p27, Protein kinase Cdelta, KV.14, Beta F1-ATPase, Cat-1, ODC, dendrin, Neurogranin/RC3, NBS1, FMR1, Rbm3, NDST (heparan sulfate/heparin GlcNAc N-deacetylase/N-sulfotransferase) as described in Vagner et al., supra 2001 and Hellen and Sarnow, supra 2001. Additional suitable IRES sequences include, for example, those set forth in GenBank Accession Nos.: NC_{—}004830; NC_{—}004004; NC_{—}003782; AJ242654; AJ242653; AJ242652; AJ242651; BD195905; BD195904; X90724; X90722; X90723; AF311318; 1F85A; 1F84A; AF308157; AB017037; E12564; Y07702; and M95781.

2. Promoters

The promoter used to direct expression of a heterologous nucleic acid depends on the particular application. The promoter is preferably positioned about the same distance from the heterologous transcription start site for the sRNAP nucleic acid as it is from the transcription start site in its natural setting. As is known in the art, however, some variation in this distance can be accommodated without loss of promoter function. The promoter typically can also include elements that are responsive to transactivation, e.g., hypoxia responsive elements, Gal4 responsive elements, lac repressor responsive elements, and the like. Examples of suitable eukaryotic promoters include a CMV promoter, a SV40 promoter, a ADH1 promoter, a GAL4 promoter, and a LexA promoter. Typically the promoter is a CMV promoter. The promoter can be constitutive (i.e., active under most environmental and developmental conditions), or inducible (i.e., active under environmental or developmental regulation), heterologous or homologous, as well as tissue-specific, or tumor-specific. Examples of suitable promoters are described in more detail below.

a) Tissue-Specific Promoters

For example, promoter sequences are known in the art that are active in specific cell types. Tissue-specific promoters have been described for endothelial cells (vWf promoter; see, e.g., Jahroudi and Lynch (1994) Mol. Cell. Biol., 14(2): 999-1008), lung epithelium (CCSP promoter; see, e.g., Stripp et al. (1994) Genomics 20(1):27-35), liver (albumin promoter; (see, e.g., Gorski et al. (1986) Cell 47(5): 767-776), bone tissue (osteoblast-specific osteocalcin promoter; (see, e.g., Lian et al. (1989) Connect. Tissue Res. 21(1-4): 61-68), and muscle (MCK promoter; see, e.g., Jaynes et al. (1988) Mol. Cell. Biol. 8(1): 62-70).

b) Tumor-Specific Promoters

In certain embodiment, the eukaryotic promoter is a tumor-specific promoter. Tumor-specific promoters are known in the art: Muc-1 promoter: Spicer et al. (1991) J. Biol. Chem. 266(23): 15099-15109, CEA promoter (see, e.g., Schrewe et al. (1990) Mol. Cell. Biol. 10(6): 2738-2748), PSA-promoter (see, e.g., Riegman et al. (1991) Mol. Endocrinol. 5(12): 1921-1930), HER-2 promoter (see, e.g., Ishii et al. (1987) Proc. Natl. Acad. Sci., U.S.A. 84(13): 4374-4378), L-plastin promoter (see, e.g., Lin et al. (1993) J. Biol. Chem. 268(4): 2793-2801), AFP promoter (see, e.g., Widen and Papaconstantinou (1986) Proc. Natl. Acad. Sci., U.S.A. 83(21): 8196-8200). These tumor-specific promoters are active in particular kinds of tumors. For example, the L-plastin promoter is active in breast cancers, the AFP promoter is active in liver tumors and the HRE promoter is active in solid tumors.

c) Inducible Promoters

In addition, there are promoters whose activity can be induced upon an external stimulus, such as the addition of an exogenous compound or upon a change in environmental conditions such as a HRE promoter (see, e.g., Dachs et al. (1997) Nat. Med. 3(5): 515-520), a Egr-1 promoter (see, e.g., Hallahan et al. (1995) Nat. Med. 1(8): 786-791), a Mdr-1 promoter (see, e.g., Ueda et al. (1987) J. Biol. Chem. 262(36): 17432-17136), a Hsp70 promoter (see, e.g., Pelham and Bienz, (1982) EMBO J. 1(11): 1473-1477), and a tetracycline-induced promoter (see, e.g., Furth et al. (1994) Proc. Natl. Acad. Sci., U. S. A. 91(20): 9302-9306. These promoters are activated with various stimuli, including radiation for the egr-1 promoter, chemotherapy for the mdr- I promoter, heat for the hsp-70 promoter and tetracycline for the tetracycline induced promoter.

3. Additional Elements

In addition to the promoter, the expression cassette typically contains a transcription unit that contains all the additional elements required for the expression of the nucleic acid in host cells. A typical expression cassette thus can contain signals required for efficient polyadenylation of the transcript, ribosome binding sites (e.g., an IRES (Internal ribosomal entry site as discussed above)), and a translation termination signal. Additional elements of the cassette may include enhancers and, if genomic DNA is used as the structural gene, introns with functional splice donor and acceptor sites.

Expression vectors containing the sRNAP expression cassette can be employed in the present invention. These vectors include SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells, such as those described above.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

4. RNAP Promoters

The expression cassettes encoding a sRNAP can also contain a RNAP promoter. In addition, the expression cassettes comprising a nucleic acid encoding a product of interest typically contain a RNAP promoter. The RNAP promoter should be recognized and competent to be transcribed by the sRNAP being employed. Preferably, the RNAP promoter is a non-host RNAP promoter. More preferably, the RNAP promoter is a phagemid promoter such as a T7 RNAP promoter, a SP6 RNAP promoter, a T3 RNAP promoter, and a K11 RNAP promoter. Examples of promoter nucleic acid sequences for phagemid RNAPs include, without limitation, TAATACGACTCACTATAGGGAGA (SEQ ID NO: 22) for T7 RNAP, ATTTAGGTGACACTATAGAAGAA (SEQ ID NO: 23) for SP6 RNAP, AATTAACCCTCACTAAAGGGAGA (SEQ ID NO: 24) for T3 RNAP, and AATTAGGGCACACTATAGGGAGA (SEQ ID NO: 25) for K11 RNAP (see e.g., Rong et al. (1999) Biotechniques 27: 690-694).

IV. Purified sRNAPs

Alternatively, the sRNAPs of the present invention can be purified from cell culture media of cells that express an sRNAP. The sRNAPs can be expressed in eukaryotic cells from a sRNAP coding sequence subcloned into a eukaryotic vector. Eukaryotic expression systems for mammalian cells, yeast, and insect cells are well known in the art and are also commercially available.

In addition, the sRNAPs of the present invention can be purified from prokaryotes. Bacterial expression systems for expressing the sRNAPs are available in, e.g., E. coli, Bacillus sp., and Salmonella (Palva et al., Gene 22:229-235 (1983)). Kits for such expression systems are commercially available. Phagemid RNAPs have been expressed in E. coli without secretion domains: T7 RNAP (Davanloo et al. (1984) Proc. Natl. Acad. Sci., U.S.A. 81: 2035-2039) and K11 RNAP (Han et al. (1999) Protein Expr. Purif. 16: 103-108).

If necessary, recombinant sRNAPs can be purified for use for use in expressing a product of interest and for preparing pharmaceutical compositions of sRNAPs. Recombinant sRNAPs can be purified from any suitable expression system, e.g., by expressing a sRNAP in E. coli and then purifying the recombinant protein via affinity purification, e.g., by using antibodies that recognize a specific epitope on the protein or on part of the fusion protein, or by using glutathione affinity gel, which binds to GST. In some embodiments, the recombinant protein is a fusion protein, e.g., a histidine tagged sRNAP, a GST tagged sRNAP, etc.

The sRNAP may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra). Preferably, the sRNAP is purified to at least 50% purity, even more preferably to at least 80% purity, still more preferably to at least 90% purity, and yet still more preferably to at least 95% purity.

A number of procedures can be employed when recombinant sRNAPs are being purified. For example, proteins having established molecular adhesion properties can be reversibly fused to a sRNAP. With the appropriate ligand, sRNAP can be selectively adsorbed to a purification column and then freed from the column in a relatively pure form. The fused protein is then removed by enzymatic activity. Finally, a sRNAP can be purified using immunoaffinity columns.

1. Purification of sRNAP from Recombinant Bacteria

Recombinant sRNAPs are expressed by transformed bacteria in large amounts, typically after promoter induction, but expression can be constitutive. Promoter induction with IPTG is one example of an inducible promoter system. Bacteria are grown according to standard procedures in the art. Fresh or frozen bacteria cells are used for isolation of protein.

sRNAPs expressed in bacteria may form insoluble aggregates (“inclusion bodies”). Several protocols are suitable for purification of inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. sRNAPs that formed the inclusion bodies may be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents which are capable of solubilizing aggregate-forming proteins, for example, SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing reformation of immunologically and/or biologically active protein. Other suitable buffers are known to those skilled in the art. The sRNAP of choice is separated from other bacterial proteins by standard separation techniques, e.g., with Ni—NTA agarose resin.

2. Standard Protein Separation Techniques for Purifying sRNAPs

a) Solubility Fractionation

Often as an initial step, particularly if the protein mixture is complex, an initial salt fractionation can separate many of the unwanted host cell proteins (or proteins derived from the cell culture media) from the recombinant sRNAP of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

b) Size Differential Filtration

The molecular weight of the protein, e.g., a sRNAP, can be used to isolated it from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant sRNAP will pass through the membrane into the filtrate. The filtrate can then be chromatographed as described below.

c) Column Chromatography

The sRNAP of choice can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

V. Expression Cassettes Encoding a Product of Interest

The expression cassettes encoding a product of interest can be on the same molecule or on a different molecule than the expression cassette encoding a sRNAP. Thus, in certain embodiments, the nucleic acid containing a sRNAP expression cassette also contains an expression cassette containing a RNA polymerase promoter operably linked to a nucleic acid encoding a product of interest. In other embodiments, the expression cassette encoding a product of interest is on a second nucleic acid molecule comprising an expression cassette containing a RNA polymerase promoter operably linked to a nucleic acid encoding a product of interest. Preferably the expression cassette encoding the therapeutic product is present on the same nucleic acid molecule as the secretable RNA polymerase expression cassette. These expression cassettes are constructed using standard molecular biology techniques similar to those used to construct the expression cassettes encoding the sRNAP.

A. Products of Interest

The RNAP promoter can be transcribed by a sRNAP that enters the cell, leading to the expression of the product of interest. The product of interest can be useful for commercial purposes, including for therapeutic purposes as a pharmaceutical or for diagnostic purposes. Some products of interest are therapeutic products. Some therapeutic products of interest (e.g., single-chain insulin, EPO) can be purified, formulated as a pharmaceutical composition and used for the treatment of a disease (e.g., diabetes, anemia, etc). In certain embodiments, the therapeutic product itself can also be a fusion protein between a secretable domain and a product of interest. Examples of therapeutic products include a protein, a nucleic acid, an antisense nucleic acid, ribozymes, tRNA, snRNA, an antigen, Factor VIII, and Apoptin (Zhuang et al. (1995) Cancer Res. 55(3): 486-489). Suitable classes of gene products include, but are not limited to, cytotoxic/suicide genes, immunomodulators, cell receptor ligands, tumor suppressors, and anti-angiogenic genes. The particular gene selected will depend on the intended purpose or treatment. Examples of such genes of interest are described below and throughout the specification.

1. Tumor Suppressors

Tumor suppressor genes are genes that are able to inhibit the growth of a cell, particularly tumor cells. Thus, delivery of these genes to tumor cells is useful in the treatment of cancers. Tumor suppressor genes include, but are not limited to, p53 (Lamb et al., Mol. Cell. Biol. 6:1379-1385 (1986), Ewen et al., Science 255:85-87 (1992), Ewen et al. (1991) Cell 66:1155-1164,and Hu et al., EMBO J. 9:1147-1155(1990)), RB1 (Toguchida et al. (1993) Genomics 17:535-543), WT1 (Hastie, N. D., Curr. Opin. Genet. Dev. 3:408-413 (1993)), NF1 (Trofatter et al., Cell 72:791-800 (1993), Cawthon et al., Cell 62:193-201 (1990)), VHL (Latif et al., Science 260:1317-1320 (1993)), APC (Gorden et al., Cell 66:589-600 (1991)), DAP kinase (see e.g., Diess et al. (1995) Genes Dev. 9: 15-30), p16 (see e.g., Marx (1994) Science 264(5167): 1846), ARF (see e.g., Quelle et al. (1995) Cell 83(6): 993-1000), Neurofibromin (see e.g., Huynh et al. (1992) Neurosci. Lett. 143(1-2): 233-236), Apoptin (Zhuang et al. (1995) Cancer Res. 55(3): 486-489), and PTEN (see e.g., Li et al. (1997) Science 275(5308): 1943-1947).

2. Immunomodulator Genes

Immunomodulator genes are genes that modulate one or more immune responses. Examples of immunomodulator genes include cytokines such as growth factors (e.g., TGF-α., TGF-β, EGF, FGF, IGF, NGF, PDGF, CGF, GM-CSF, G-CSF, SCF, etc.), interleukins (e.g., IL-2, IL-3, IL-4, IL-6, IL-7, IL-10, IL-12, IL-15, IL-20, etc.), interferons (e.g., IFN-α, IFN-β, IFN-γ, etc.), TNF (e.g., TNF-α), and Flt3-Ligand.

3. Cell Receptor Ligands

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g,. inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, single-chain insulin (Lee et al. (2000) Nature 408: 483-488), glucagon, G-protein coupled receptor ligands, etc.). These cell surface ligands can be useful in the treatment of patients suffering from a disease. For example, a single-chain insulin when expressed under the control of the glucose-responsive hepatocyte-specific L-type pyruvate kinase (LPK) promoter was able to cause the remission of diabetes in streptocozin-induced diabetic rats and autoimmune diabetic mice without side effects (Lee et al. (2000) Nature 408:483-488). This single-chain insulin was created by replacing the 35 amino acid resides of the C-peptide of insulin with a short turn-forming heptapeptide (Gly-Gly-Gly-Pro-Gly-Lys-Arg).

4. Anti-Angiogenic Genes

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see e.g., U.S. Pat. No. 6,174,861), angiostatin (see, e.g., U.S. Pat. No. 5,639,725), and VEGF-R2 (see e.g., Decaussin et al. (1999) J. Pathol. 188(4): 369-737).

5. Cytotoxic/Suicide Genes

Cytotoxic/suicide genes are those genes that are capable of directly or indirectly killing cells, causing apoptosis, or arresting cells in the cell cycle. Such genes include, but are not limited to, genes for immunotoxins, a herpes simplex virus thymidine kinase (HSV-TK), a cytosine deaminase, a xanthine-guaninephosphoribosyl transferase, a p53, a purine nucleoside phosphorylase, a carboxylesterase, a deoxycytidine kinase, a nitroreductase, a thymidine phosphorylase, and a cytochrome P450 2B 1.

In a gene therapy technique known as gene-delivered enzyme prodrug therapy (“GDEPT”) or, alternatively, the “suicide gene/prodrug” system, agents such as acyclovir and ganciclovir (for thymidine kinase), cyclophosphoamide (for cytochrome P450 2B1), 5-fluorocytosine (for cytosine deaminase), are typically administered systemically in conjunction (e.g., simulatenously or nonsimulatenously, for example, sequentially) with a expression cassette encoding a suicide gene compositions of the present invention to achieve the desired cytotoxic or cytostatic effect (see, e.g., Moolten, F. L., Cancer Res., 46:5276-5281 (1986)). For a review of the GDEPT system, see, Moolten, F. L., The Internet Book of Gene Therapy, Cancer Therapeutics, Chapter 11 (Sobol, R. E., Scanlon, N.J. (Eds) Appelton & Lange (1995)). In this method, a heterologous gene is delivered to a cell in an expression cassette containing a RNAP promoter, the heterologous gene encoding an enzyme that promotes the metabolism of a first compound to which the cell is less sensitive (i.e., the “prodrug”) into a second compound to which is cell is more sensitive. The prodrug is delivered to the cell either with the gene or after delivery of the gene. The enzyme will process the prodrug into the second compound and respond accordingly. A suitable system proposed by Moolten is the herpes simplex virus-thymidine kinase (HSV-TK) gene and the prodrug ganciclovir. This method has recently been employed using cationic lipid-nucleic aggregates for local delivery (i.e., direct intra-tumoral injection), or regional delivery (i.e., intra-peritoneal) of the TK gene to mouse tumors by Zerrouqui, et al., Can. Gen. Therapy, 3(6):385-392 (1996); Sugaya, et al., Hum. Gen. Ther., 7:223-230 (1996) and Aoki, et al., Hum. Gen. Ther., 8:1105-1113 (1997). Human clinical trials using a GDEPT system employing viral vectors have been proposed (see, Hum. Gene Ther., 8:597-613 (1997), and Hum. Gene Ther., 7:255-267 (1996)) and are underway.

For use with the instant invention, the most preferred therapeutic products are those which are useful in gene-delivered enzyme prodrug therapy (“GDEPT”). Any suicide gene/prodrug combination can be used in accordance with the present invention. Several suicide gene/prodrug combinations suitable for use in the present invention are cited in Sikora, K. in OECD Documents, Gene Delivery Systems at pp.59-71 (1996), incorporated herein by reference, include, but are not limited to, the following:

| Suicide Gene Product | Less Active ProDrug | Activated Drug |
|---|---|---|
| Herpes simplex virus | ganciclovir(GCV), | phosphorylated |
| type 1 thymidine | acyclovir, | dGTP analogs |
| kinase (HSV-TK) | bromovinyl- | |
| | deoxyuridine, or other | |
| | substrates | |
| Cytosine Deaminase | 5-fluorocytosine | 5-fluorouracil |
| (CD) | | |
| Xanthine-guanine- | 6-thioxanthine (6TX) | 6-thioguano- |
| phosphoribosyl | | sinemonophosphate |
| transferase (XGPRT) | | |
| Purine nucleoside | MeP-dr | 6-methylpurine |
| phosphorylase | | |
| Cytochrome P450 | cyclophosphamide | [cytotoxic |
| 2B1 | | metabolites] |
| Linamarase | amygdalin | cyanide |
| Nitroreductase | CB 1954 | nitrobenzamidine |
| Beta-lactamase | PD | PD mustard |
| Beta-glucuronidase | adria-glu | adriamycin |
| Carboxypeptidase | MTX-alanine | MTX |
| Glucose oxidase | glucose | peroxide |
| Penicillin amidase | adria-PA | adriamycin |
| Superoxide dismutase | XRT | DNA damaging agent |
| Ribonuclease | RNA | cleavage products |

Any prodrug can be used if it is metabolized by the heterologous gene product into a compound to which the cell is more sensitive. Preferably, cells are at least 10-fold more sensitive to the metabolite than the prodrug.

Modifications of the GDEPT system that may be useful with the invention include, for example, the use of a modified TK enzyme construct, wherein the TK gene has been mutated to cause more rapid conversion of prodrug to drug (see, for example, Black, et al., Proc. Natl. Acad. Sci, U.S.A., 93: 3525-3529 (1996)). Alternatively, the TK gene can be delivered in a bicistronic construct with another gene that enhances its effect. For example, to enhance the “bystander effect” also known as the “neighbor effect” (wherein cells in the vicinity of the transfected cell are also killed), the TK gene can be delivered with a gene for a gap junction protein, such as connexin 43. The connexin protein allows diffusion of toxic products of the TK enzyme from one cell into another. The TK/Connexin 43 construct has a CMV promoter operably linked to a TK gene by an internal ribosome entry sequence and a Connexin 43-encoding nucleic acid.

VI. Methods for Introducing Expression Cassettes Into Cells

Methods are well known in the art for introducing nucleic acids into cells. (see, e.g., CURRENT PROTOCOLS IN MOLECULAR BIOLOGY (Ausubel, et al. (eds.) 1995). These methods can be used to introduce into cells a nucleic acid containing an expression cassette comprised of a RNA polymerase promoter operably linked to a nucleic acid encoding a product of interest, as well as an expression cassette encoding a sRNAP. The expression cassettes can be introduced into the same cell on the same molecule, into the same cell on different molecules, into different cells on two different molecules, etc. Methods such as biollistics, transfection, electroporation, viral delivery systems, etc. can be employed in the present invention. In addition, the nucleic acids can be formulated using a variety of compounds known in the art for packaging nucleic acids for introduction into cells, such as polylysine, polyethylenimine (PEI), DEAE-dextran, and lipids.

In preferred embodiments, the nucleic acids of the present invention are delivered into cells as a lipid-nucleic acid composition containing a nucleic acid-lipid particle comprising a lipid portion and a nucleic acid portion. In particularly preferred embodiments the lipid-nucleic acid composition is a stabilized-stable lipid particle, wherein the nucleic acid is fully encapsulated within said lipid portion (see, e.g., Wheeler et al. (1999) Gene Therapy 6: 271-281). Preferred lipids include those protonatable lipids having a pKa in a range of about 4 to about 11. Cationic lipids are also useful in formulating the lipid portion of the composition. The cationic lipid can comprise varying mole percents of the lipid portion. Examples of cationic lipids include, without limitation, DODAC, DODAP, DODMA, DOTAP, DOTMA, DC-Chol, DMRIE, and DSDAC. Non-cationic lipids are also useful in formulating the lipid portion of the composition. The non-cationic lipid can comprise varying mole percents of the lipid portion. Examples of non-cationic lipids include, without limitation, phospholipid-related materials, such as lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl- phosphatidylethanolamine (POPE) and dioleoyl- phosphatidylethanolamine 4-(N-maleimidomethyl)-cyclohexane-1-carboxylate (DOPE-mal). Noncationic lipids or sterols such as cholesterol may be present. Additional nonphosphorous containing lipids are, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Other lipids such as lysophosphatidylcholine and lysophosphatidylethanolamine may be present. Noncationic lipids also include polyethylene glycol-based polymers such as PEG 2000, PEG 5000 and polyethylene glycol conjugated to phospholipids or to ceramides (referred to as PEG-Cer), as described in co-pending U.S. Ser. No. 08/316,429, incorporated herein by reference.

Moreover, the lipid-therapeutic nucleic acid particles of the present invention are serum-stable and, thus, not significantly degraded after exposure to a serum or nuclease assay that would significantly degrade free DNA. Suitable assays for measuring serum stability include a standard serum assay or a DNase assay (which are described in the Example section). Nuclease resistance/serum stability is a measure of the ability of the formulation to protect the therapeutic nucleic acid from nuclease digestion either in an in vitro assay or in circulation. The encapsulated particles of the present invention have greater nuclease resistance and serum stability than lipid-plasmid aggregates (also known as cationic complexes or lipoplexes), such as DOTMA/DOPE (LIPOFECTIN™) formulations.

In addition, the lipid-therapeutic nucleic acid particles of the present invention have a nucleic acid to lipid ratio that can be formulated at various levels. For use in the methods of this invention, the particles have a drug to lipid ratio of at least about 3 mg of nucleic acid per mmol of lipid, more preferably, at least about 14 mg of nucleic acid per mmol of lipid and, even more preferably, greater than about 25 mg of nucleic acid per mmol of lipid. The preferred particles, when prepared to an administration ready formulation, are about 60-80 mg nucleic acid per mmol lipid (i.e., they are “high ratio” formulations). The method used for making high ratio formulations can also be employed using reduced amounts of DNA to obtain lower ratio formulations. As used herein, “drug to lipid ratio” refers to the amount of therapeutic nucleic acid (i.e., the amount of nucleic acid that is encapsulated and that will not be rapidly degraded upon exposure to the blood) in a defined volume of preparation divided by the amount of lipid in the same volume. This may be determined on a mole per mole basis, on a weight per weight basis, or on a weight per mole basis. For final administration ready formulations, the drug to lipid ratio is calculated after dialysis, chromatography and/or nuclease digestion have been employed to remove as much of the externally associated therapeutic agent as possible. Drug to lipid ratio is a measure of potency of the formulation, although the highest possible drug to lipid ratio is not always the most potent formulation.

An alternative description of the lipid-nucleic acid particles of the present invention is “high efficiency” formulations that emphasizes the active loading process involved and contrasts with low efficiency or passive encapsulation. Passive encapsulation of nucleic acid in lipid particles, which is known in the art, achieves less than 15% encapsulation of therapeutic agent, and results in low ratio particles having less than 3 mg of nucleic acid per mmol of lipid. The preferred lipid/therapeutic nucleic acid particles of the present invention have an encapsulation efficiency of greater than about 30%. As used herein, “encapsulation efficiency” refers to absolute efficiency, i.e., the total amount of DNA added to the starting mixture that ends up in the administration competent formulation. Sometimes the relative efficiency is calculated, wherein the drug to lipid ratio of the starting mixture is divided by the drug to lipid ratio of the final, administration competent formulation. The amount of lipid lost during the formulation process may be calculated. Efficiency is a measure of the wastage and expense of the formulation.

Other beneficial features that flow from the use of the preferred particles of the present invention, such as low nonspecific toxicity, improved biodistribution, therapeutic efficacy and ease of manufacturing, will be apparent to those of skill in the art. It is possible to develop particles as described above by alternative methods of encapsulation. These methods may employ standard techniques for loading of liposomes that are well known for use with conventional drugs. These methods include freeze-thaw extrusion, dehydration/rehydration, reverse phase evaporation, and the like, some of which are disclosed in Monnard, et al., “Entrapment of nucleic acids in liposomes, “Biochim. Biophys. Acta., 1329:39-50 (1997). These methods are not high encapsulation efficiency formulations, nor high ratio formulations, but the instant disclosure suggests the utility of such particles in the use of gene therapy against distal tumor sites.

In addition to the lipids employed in the methods used above, there are a tremendous number of additional lipid and nonlipid components which can be used to enhance delivery or targeting of particles. Additional lipid components include, but are not limited to, lipids with neutral, anionic, cationic or zwitterionic headgroups, and the like. These standard components are set out in the art and in the patent applications referred to above which are incorporated herein by reference. Charged lipids that are particularly preferred with the invention are N,N-dioleyl-N,N-dimethylammonium chloride (DODAC), the subject of recently issued U.S. Pat. No. 5,753,613, incorporated herein by reference; and 1,2-Dioleoyl-3-dimethylammonium-propane (DODAP), the subject of U.S. patent application Ser. No. 08/856,374, the teachings of which are incorporated herein by reference.

In addition, cloaking agents or bilayer stabilizing compents can be used to reduce elimination by the host immune system. Such cloaking agents include, for example, polyamide oligomer-lipid conjugates, such as ATTA-lipids, disclosed in U.S. patent application Ser. No. 08/996,783, filed Feb. 2, 1998 and PEG-lipid conjugates (e.g., PEG-ceramides, PEG-phohspholipids, and PEG-diacylglycerols), some of which are disclosed in U.S. patent application Ser. Nos. 08/486,214, 08/316,407, 08/485,608, and 10/136,707 the teachings of which are incorporated herein by reference. These components can also be targeting agents that encourage the lipid formulations to accumulate at the area of the disease or target site. In addition, these components can be compounds that improve features of the formulation, such as leakiness, longevity in circulation, reduction in toxicity, encapsulation efficiency, etc. Examples of these components and others that can usefully be included in the formulations of the invention are known to and used by those skilled in the art.

VII. Methods of Expressing a Nucleic Acid Encoding a Product of Interest

The expression cassettes encoding a product of interest can be expressed in a cell using the methods of the present invention. In one embodiment, the product of interest is expressed in a cell by introducing into the cell an expression cassette comprised of a RNA polymerase promoter operably linked to a nucleic acid encoding a product of interest. The cell is then contacted with a sRNAP. Methods for introducing nucleic acids into cells have been described above. The sRNAP can be produced by another cell or bacteria, purified and then contacted with the cell containing the product of interest expression cassette. In other embodiments, the sRNAP is expressed from a cell in the same cell culture medium that is in contact with the cell containing the product of interest expression cassette. The sRNAPs when contacted with a cell, are taken up by that cell into the cytoplasm. The sRNAP will then transcribe the expression cassette encoding the product of interest. If the product of interest is a pharmaceutical, such as insulin or EPO, then it can be purified and processed for human clinical use to treat diseases such as diabetes (insulin) and anemia (EPO). Products of interest such as a restriction endonuclease can also be produced to be used in molecular biology techniques that are useful for diagnosing diseases (e.g., RFLP, etc.). In a preferred embodiments the product of interest is expressed by introducing into the cell an expression cassette encoding the product of interest present on the same nucleic acid molecule as the secretable RNA polymerase expression cassette.

VIII. Methods of Treating Disease

In certain embodiments, the methods of the present invention involve treating a disease in a subject. Essentially any disease that can be treated that involves the delivery of a therapeutic product to a situs involved in the pathology of a disease. In certain embodiments, cancers can be treated using the methods of the present invention. Cancers include without limitation, cancers of the brain, lung, prostate, breast, bone, pancrease, liver, kidney, mouth, ears, nose, throat, skin, colon, and blood. In addition autoimmune diseases such as myasthenia gravis (MG), systemic lupus erythematosis (SLE), rheumatoid arthritis (RA), multiple sclerosis (MS), and insulin-dependent diabetes mellitus (IDDM), can be treated using the methods of the present invention. Also, diseases such as cardiovascular diseases (e.g., hypercholesterolemia, hypertension, congestive heart failure, atherosclerosis, etc.), cystic fibrosis, sickle cell anemia, hemophilia, infectious disease (viral disease (AIDS, Herpes, etc), bacterial (pneumonia, TB, etc), and inflammatory diseases.

The methods of treating these diseases involve administering a therapeutically effective amount of an expression cassette comprised of a RNA polymerase promoter operably linked to a nucleic acid encoding a therapeutic product; and administering a therapeutically effective amount of a secretable RNA polymerase, wherein the secretable RNA polymerase comprises a RNA polymerase and a secretion domain. The expression cassette encoding a therapeutic product and the secretable RNA polymerase expression cassette can be present on the same or different molecules, preferably on the same molecule. In other embodiments, the sRNAP can be delivered as a purified sRNAP.

In particularly preferred embodiments, a cancer is treated by administering a sRNAP and an expression cassette encoding a cytotoxic gene that can convert a prodrug into a toxic compound, which is a version of the GDEPT system. The sRNAP and the therapeutic product expression cassette can be delivered simultaneously or non-simultaneously, preferably on the same molecule. The prodrug is then delivered as the free drug or, alternatively, it can be in a lipid formulation. Usually, the expression cassette encoding the therapeutic product will be delivered with the sRNAP to the target cell in advance of the prodrug in order to allow synthesis of the suicide gene product prior to the arrival of the prodrug. Thus, using the compositions and methods of the invention, the therapeutic product is delivered to the cell to direct synthesis of the suicide gene product, the cell is thereby sensitized, the prodrug is delivered to the cell, and patient therapy, e.g., reduction of tumor size, inflammation or infectious load and the like, is achieved.

Combinations of expression cassettes, sRNAPs that are useful for treating cancers can be assayed for their effects on cell growth. If the product of interest is a product that can be used to treat cancer or to inhibit the growth of a cell, then a variety of in vitro and in vivo assays can be used to assess whether the product of interest is effective, e.g., ability to grow on soft agar, changes in contact inhibition and density limitation of growth, changes in growth factor or serum dependence, changes in the level of tumor specific markers, changes in invasiveness into Matrigel, changes in tumor growth in vivo, such as in transgenic mice, etc.

A. Assays for Changes in Cell Growth by Expression of Product of Interest Constructs

The following are assays that can be used to identify product of interest constructs which are capable of regulating cell proliferation and tumor suppression. Functional product of interest constructs identified by the following assays can then be used in gene therapy to inhibit abnormal cellular proliferation and transformation.

1. Soft Agar Growth or Colony Formation in Suspension

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Soft agar growth or colony formation in suspension assays can be used to identify product of interest constructs, which when expressed in host cells, inhibit abnormal cellular proliferation and transformation. Typically, transformed host cells (e.g., cells that grow on soft agar) are used in this assay. Expression of a tumor suppressor gene in these transformed host cells would reduce or eliminate the host cells' ability to grow in stirred suspension culture or suspended in semi-solid media, such as semi-solid or soft. This is because the host cells would regenerate anchorage dependence of normal cells, and therefore require a solid substrate to grow. Therefore, this assay can be used to identify product of interest constructs which function as a tumor suppressor. Once identified, such product of interest constructs can be used in gene therapy to inhibit abnormal cellular proliferation and transformation.

Techniques for soft agar growth or colony formation in suspension assays are described in Freshney, Culture of Animal Cells a Manual of Basic Technique, 3^{rd} ed., Wiley-Liss, New York (1994), herein incorporated by reference. See also, the methods section of Garkavtsev et al. (1996), supra, herein incorporated by reference.

2. Contact Inhibition and Density Limitation of Growth

Normal cells typically grow in a flat and organized pattern in a petri dish until they touch other cells. When the cells touch one another, they are contact inhibited and stop growing. When cells are transformed, however, the cells are not contact inhibited and continue to grow to high densities in disorganized foci. Thus, the transformed cells grow to a higher saturation density than normal cells. This can be detected morphologically by the formation of a disoriented monolayer of cells or rounded cells in foci within the regular pattern of normal surrounding cells. Alternatively, labeling index with [³H]-thymidine at saturation density can be used to measure density limitation of growth. See Freshney (1994), supra. The transformed cells, when transfected with tumor suppressor genes, regenerate a normal phenotype and become contact inhibited and would grow to a lower density.

Contact inhibition and density limitation of growth assays can be used to identify product of interest constructs which are capable of inhibiting abnormal proliferation and transformation in host cells. Typically, transformed host cells (e.g., cells that are not contact inhibited) are used in this assay. Expression of a tumor suppressor gene in these transformed host cells would result in cells which are contact inhibited and grow to a lower saturation density than the transformed cells. Therefore, this assay can be used to identify product of interest constructs which function as a tumor suppressor. Once identified, such product of interest constructs can be used in gene therapy to inhibit abnormal cellular proliferation and transformation.

In this assay, labeling index with [³H]-thymidine at saturation density is a preferred method of measuring density limitation of growth. Transformed host cells are transfected with a product of interest construct and are grown for 24 hours at saturation density in non-limiting medium conditions. The percentage of cells labeling with [³H]-thymidine is determined autoradiographically. See, Freshney (1994), supra. The host cells expressing a functional product of interest construct would give arise to a lower labeling index compared to control (e.g., transformed host cells transfected with a vector lacking an insert).

3. Growth Factor or Serum Dependence

Growth factor or serum dependence can be used as an assay to identify functional product of interest constructs. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When a tumor suppressor gene is transfected and expressed in these transformed cells, the cells would reacquire serum dependence and would release growth factors at a lower level. Therefore, this assay can be used to identify product of interest constructs which function as a tumor suppressor. Growth factor or serum dependence of transformed host cells which are transfected with a product of interest construct can be compared with that of control (e.g., transformed host cells which are transfected with a vector without insert). Host cells expressing a functional product of interest would exhibit an increase in growth factor and serum dependence compared to control.

4. Tumor-Specific Marker Levels

Tumor cells release an increased amount of certain factors (hereinafter “tumor-specific markers”) than their normal counterparts. For example, plasminogen activator (PA) is released from human glioma at a higher level than from normal brain cells (see, e.g., Gullino, Angiogenesis, tumor vascularization, and potential interference with tumor growth. In Mihich (ed.): “Biological Responses in Cancer.” New York, Academic Press, pp. 178-184 (1985)). Similarly, Tumor angiogenesis factor (TAF) is released at a higher level in tumor cells than their normal counterparts. See, e.g., Folkman, Angiogenesis and cancer, Sem Cancer Biol. (1992)).

Tumor-specific markers can be assayed for to identify product of interest constructs, which when expressed, decrease the level of release of these markers from host cells. Typically, transformed or tumorigenic host cells are used. Expression of a tumor suppressor gene in these host cells would reduce or eliminate the release of tumor-specific markers from these cells. Therefore, this assay can be used to identify product of interest constructs which function as a tumor suppressor.

Various techniques which measure the release of these factors are described in Freshney (1994), supra. Also, see, Unkless et al. , J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

5. Invasiveness into Matrigel

The degree of invasiveness into Matrigel or some other extracellular matrix constituent can be used as an assay to identify product of interest constructs which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used as host cells. Expression of a tumor suppressor gene in these host cells would decrease invasiveness of the host cells. Therefore, functional product of interest constructs can be identified by measuring changes in the level of invasiveness between the host cells before and after the introduction of product of interest constructs. If a product of interest construct functions as a tumor suppressor, its expression in tumorigenic host cells would decrease invasiveness.

Techniques described in Freshney (1994), supra, can be used. Briefly, the level of invasion of host cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

6. Cell Cycle Analysis

Cell cycle analysis can be used to determine if a gene can suppress the growth of a cell. Briefly, cells are transfected with an expression cassette containing the gene of interest. If the gene encodes a protein or other gene product that can arrest or inhibit cell division then the gene is suppressing the growth of the cells. Cell division, or mitosis, consists of several successive phases in a eukaryotic cell (Molecular Biology of the Cell, 3d edition (Alberts et al., eds., 1994)). These phases, in order, are known as G₁, S, G₂ and M. DNA replication takes place during the S phase. The mitotic phase, where nuclear division takes place, is termed the M phase. The G₁ phase is the time between the M phase and the S phase. G₂ is the time between the end of the S phase and the beginning of the M phase. Cells can pause in G₁ and enter a specialized resting state known as G₀. Cells can remain in G₀ for days to years, until they resume the cell-cycle. Methods of analyzing the phase of the cell-cycle are known in the art and include methods that involve determining if the cell is replicating DNA (e.g., [H³]-thymidine incorporation assays). Alternatively, methods are known in the art for measuring the DNA content of a cell, which doubles during the S phase. FACS (Fluorescent activated cell sorting) analysis can be used to determine the percentage of a population of cells in a particular stage of the cell-cycle (see generally, Alberts et al., supra; see also van den Heuvel and Harlow, (1993) Science 262: 2050-2054). The cells are incubated with a dye that fluoresces (e.g., propidium iodide) when it binds to the DNA of the cell. Thus, the amount of fluorescence of a cell is proportional to the DNA content of a cell. Cells that are in G₁ or G₀ (G₁/G₀) have an unreplicated complement of DNA and are deemed to have 1 arbitrary unit of DNA in the cell. Those cells that have fully replicated, i.e., have doubled their DNA content, are deemed to have 2 arbitrary units of DNA in the cell and are in the G₂ or M phase (G₂/M) of the cell cycle. Cells with an amount of DNA that is between 1 and 2 arbitrary units are in S phase.

The effect of a protein of interest on the cell cycle can be determined by transfecting cells with DNA encoding the protein of interest and analyzing its effect on the cell cycle through flow cytometry in a FACS. The cells are co-transfected with a vector encoding a marker to identify and analyze those cells that are actually transfected. Such markers can include the B cell surface marker CD20 (van de Heuvel and Harlow, supra) or a farnesylated green fluorescent protein (GFP-F) (Jiang and Hunter, (1998) Biotechniques, 24(3): 349-50, 352, 354).

For example, the percentage of cells in a particular stage of the cell-cycle can be determined using the method of Jiang and Hunter, (1998) supra. Briefly, a population of cells are transfected with a vector encoding a product of interest and a vector encoding a green fluorescent protein (GFP) with a famesylation signal sequence from c-Ha-Ras. The famesylation signal sequence is famesylated in the cell, which targets the GFP molecule to the plasma membrane. Vectors encoding famesylated GFP are commercially available (e.g., pEGFP-F from Clontech).

After transfection, the cells are suspended in buffer containing the DNA intercalator propidium iodide. Propidium iodide will fluoresce when it is bound to DNA. Thus, the amount of fluorescence observed from propidium iodide in a FACS flow cytometer is an indication of the DNA content of a cell. The percentages of cells in each cell cycle can be calculated using computer programs, e.g., the ModFit program (Becton-Dickinson). The cell cycle stage of the cell was analyzed after gating cells by GFP fluorescence using FACscan. If the gene encodes a tumor suppressor, the percentage of cells that enter S phase would be decreased, as the cells are arrested in the G₀/G₁ phase. Therefore, the percentage of cells that are G₀/G₁ phase would be increased.

IX. Administration-Ready Pharmaceutical Preparations

Generally, when administered intravenously, the nucleic acid and/or the prodrug formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration. Carriers may also be employed when delivering the vector or prodrug formulations by other parenteral methods known in the art, such as subcutaneous, intratumoral or intramuscular injection, inhalation, and the like.

When preparing pharmaceutical preparations of the lipid/therapeutic nucleic acid particles of the invention, it is preferable to use quantities of the particles which have been purified to reduce or eliminate empty particles or particles with nucleic acid associated with the external surface.

A. Modes of Administration

The nucleic acids, sRNAPs, compounds, and compositions of the present invention can be delivered to treat disease in a subject, typically a mammalian subject (e.g., a bovine, canine, feline, equine, or human subject, preferably a bovine or human subject, more preferably a human subject), using methods and modes of administration known to those of skill in the art. Suitable modes of administration include, for example, intra-cranial, intraperitoneal, intramuscular, intravenous, subcutaneous, oral, topical, and the like. In certain embodiments, the sRNAP can be delivered to the subject at a site distal to a site where the product of interest is administered due to the translocation properties of the sRNAP. In other embodiments, the therapeutic product also has a secretion domain and can cross the blood-brain barrier. In certain embodiments, where a cancer is being treated, the nucleic acids, sRNAPs, compounds, and/or compositions can be injected, for example, intravenously into blood veins feeding the tumor mass, or directly into the tumor (e.g., intratumoral injection).

### EXAMPLES

The following examples are offered to illustrate, but not to limit the claimed invention.

### Example 1

### In vitro Transcription and Translation of Secretable RNAP

A secretable RNAP expression cassette is added to an expression cassette encoding a reporter gene or product of interest and a RNA polymerase. Reporter gene activity is measured or the product of interest is detected. The reporter gene or product of interest is expressed if the RNAP polymerase transcribes the secretable RNAP expression cassette into mRNA. The secretable RNAP then transcribes the expression cassette encoding the reporter gene or the product of interest.

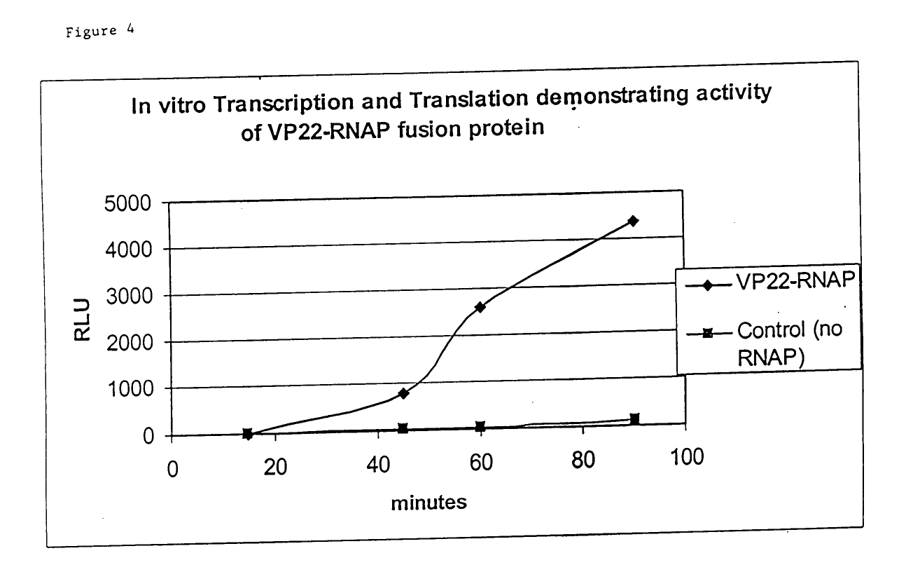

500 ng of a SP6-VP22-T7-RNAP (VP22: SEQ ID NO:21) construct was added to 250 ng of a T7-luciferase construct and 1 μl of SP6 RNA polymerase. Luciferase activity was measured over time. The results are shown in FIG. 4.

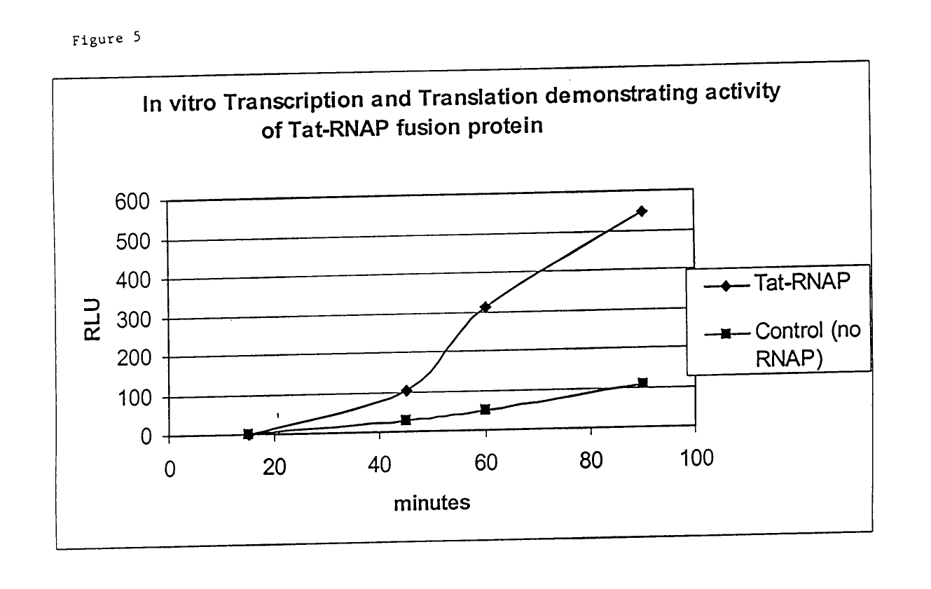

500 ng of a SP6-Tat-T7-RNAP (Tat: SEQ ID NO:1) construct was added to 250 ng of a T7-luciferase construct and 1 μl of SP6 RNA polymerase. Luciferase activity was measured over time. The results are shown in FIG. 5.

### Example 2

### Transfection of Cells with Secretable RNAP

Cells are transfected with an expression cassette encoding a reporter gene or a product of interest and an expression cassette encoding a secretable RNA polymerase. Transfection may be simultaneous or sequential. The expression cassettes may be naked nucleic acid or may be encapsulated in a liposome. Cells are harvested at several time points after transfection. Reporter gene activity is measured or the product of interest is detected.

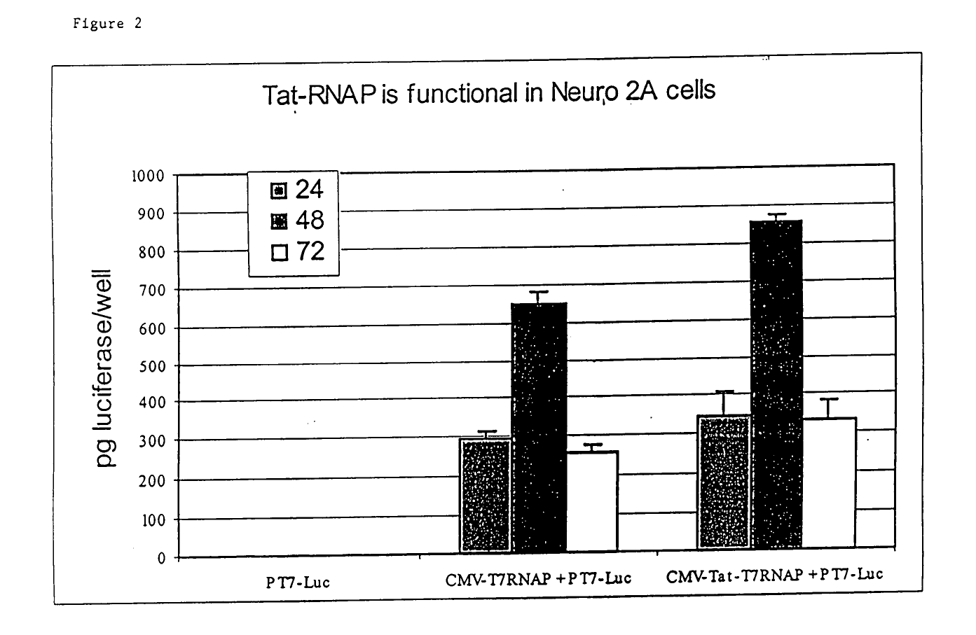

Neuro 2A cells were transfected with T7-luciferase and CMV-Tat-RNAP (Tat: SEQ ID NO:1) constructs in DOPE;DODAC (50:50) large unilamellar vesicles (LUVs).Cells were harvested 24, 48, and 72 hours after transfection and luciferase activity was measured. The results are shown in FIG. 2.

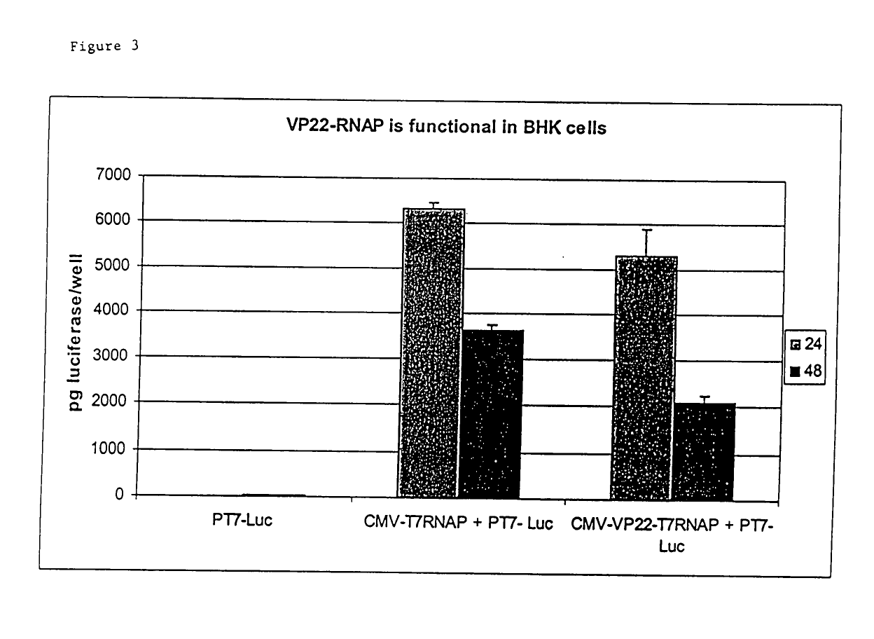

BHK cells were transfected with T7-luciferase and CMV-VP22-RNAP (VP22: SEQ ID NO:21) constructs in DOPE;DODAC (50:50) large unilamellar vesicles (LUVs). Cells were harvested 24, 48, and 72 hours after transfection and luciferase activity was measured. The results are shown in FIG. 3.

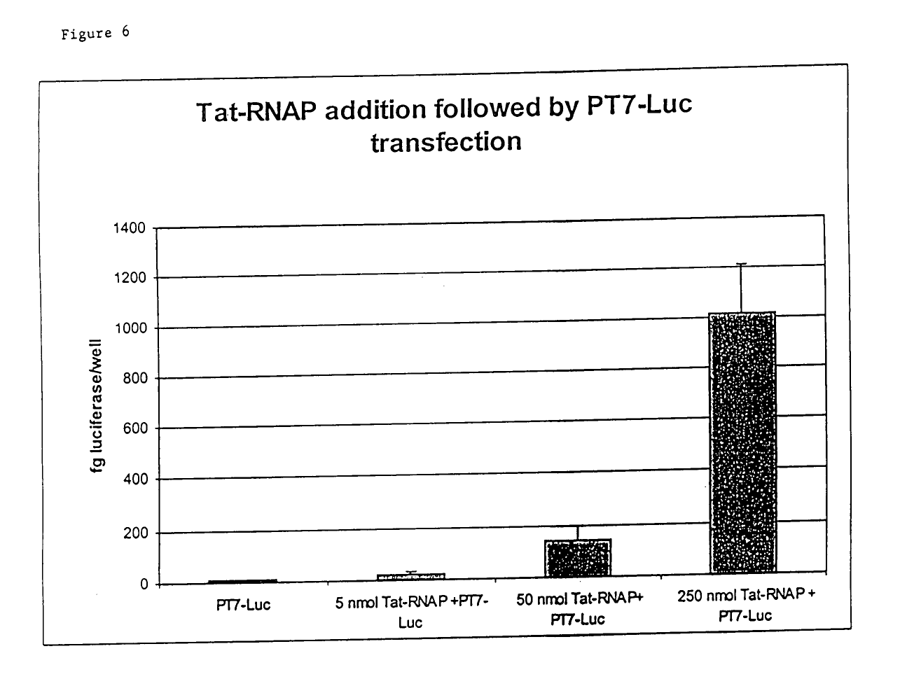

BHK cells were transfected with 5, 50, or 250 nmol of purified Tat-RNAP (Tat: SEQ ID NO: 1) for 4 hours, washed with PBS, and transfected with 0.75 μg of a T7-luciferase construct. Luciferase activity was measured. The results are shown in FIG. 6.

### Example 3

### Transfection of Cells with Secretable RNAP

Cells are transfected with an expression cassette encoding a reporter gene or an expression cassette encoding a product of interest and a secretable RNA polymerase. The expression cassette may be naked nucleic acid or may be encapsulated in a liposome at suitable times after transfection, cell populations are mixed. Cells are harvested at several time points after mixing. Reporter gene activity is measured or the product of interest is detected.

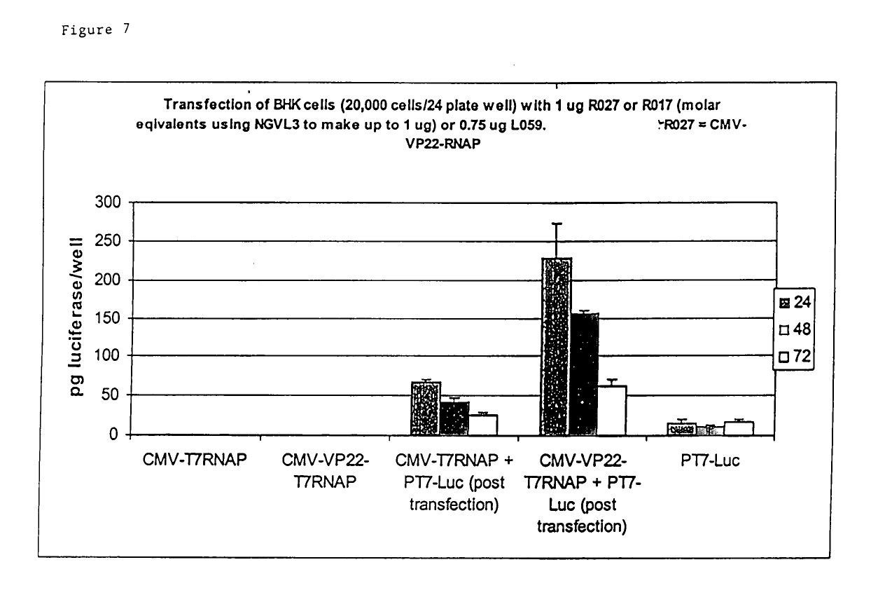

BHK cells were transfected with 1 μg of a CMV-T7 RNAP construct or a CMV-VP22-T7RNAP construct (VP22: SEQ ID NO:21). Four hours after transfection, the BHK cells were trypsinized and added to BHK cells transfected with T7-luciferase. Cells were harvested 24, 48, or 72 hours after mixing of the cell populations and luciferase activity was measured. The results are shown in FIG. 7.

### Example 4

### DNAse I Assay

To evaluate the protective effect of the lipid on nucleic acids, the nucleic acid-lipid particle is incubated with DNase I at a concentration where the nucleic acid alone is susceptible to degradation at 37° C. for 10 minutes. The reaction is stopped by the addition of 25 mM EDTA and the samples are extracted using methods known in the art, in the presence of 150 mM NaCl. (See, e.g., Bligh and Dyer, Ca. J. Biochem. Physiol. 37:91 (1959)). The DNA is precipitated with 1/10^{th} volume of 3 M sodium acetate (pH 5.2) and 2.5 volumes of 95% EtOH and recovered by centrifugation at 14,000×G for 30 minutes at 4° C. The DNA pellet is resuspended in sterile distilled water and subjected to electrophoresis on an 0.8% agarose gel.

### Example 5

### Serum Stability Assay

To evaluate the serum stability of the nucleic acid-lipid particles, an aliquot of the nucleic acid-lipid particle is incubated in mouse serum 37° C. for 30 minutes. The incubation mixture is eluted in HBS on a Sepharose CL-4B column. Comigration of the nucleic acid and lipid in the void volume suggests that no nucleic acid degradation has occurred.

### Example 6

### Materials and Methods

Plasmids and Primers: Plasmid R023 comprises a basic autogene cassette driven by a CMV promoter and intron. The autogene cassette was derived from the plasmid T7-G1, a gift of Dr. Jon Wolff (Waisman Center, Wis.). T7-G1 contains the basic autogene cassette, comprising the T7 promoter, EMCV IRES, and T7 RNAP gene. The nuclear localization sequence was removed from the T7 RNAP via PCR prior to subcloning into R023. L059 comprises a pTRI-Amp (Ambion) backbone with EMCV IRES, Photinus pyralis luciferase and beta-globin poly-adenylation site derived from EMC-Luc (Jon Wolff). L053 consists of the CMV promoter (with intron) from NGVL3 and the Photinus pyralis luciferase gene. L069 and L070 comprises L053 containing one or two irrelevant 2.5 kb spacer fragments respectively. R037 comprises R023 without the T7 and T3 promoters. R011 is a bi-cistronic plasmid comprising R023 with a downstream luciferase reporter gene cassette from L059 (bi-cistronic). PT7-Luc (Promega) comprises the Photinus pyralis luciferase gene driven by a T7 RNAP promoter. RPA-RNAP comprises a 350 bp Kpn I—Afl II T7 RNAP fragment blunted and ligated into the Sma I site of pTRI-Amp in reverse orientation. RPA-Luc comprises a 250 bp Xcm1—BsrG 1 luciferase fragment blunted and ligated into the Smal site of pTRI-Amp in the reverse orientation.

The NVSC1 primer sequence is 5′-TCCTGCAGCCCGGGGGATCCTCTAG-3′.

The resulting RO11 construct comprises the following components: a CMV promoter from about base 93 to about base 681; a first eukaryotic promoter from about base 1298 to about base 1376; a first ECMV IRES from about base 1448 to about base 2030; a nucleic acid sequence encoding RNAP from about base 2033 to about base 4681; a second eukaryotic promoter from about base 5241 to about base 5319; a second ECMV IRES from about base 5378 to about base 5963; and a nucleic acid encoding a gene of interest (e.g., a marker gene such as, for example, luciferase) from about base 5965 to about base 5963.

Transcription and Translation Assays: A 25 μl reaction was set up using a Promega (Wisconsin) Coupled In Vitro Transcription and Translation kit as per the manufacturers instructions. 250 ng of PT7-Luc was added to all reactions. 250 ng of either R023 (containing a T7 RNAP gene driven by the T7, SP6 and T3 promoters) or R037 (containing a T7 RNAP gene driven only by the SP6 promoter) was then added to the reactions. 0.5 U of SP6 RNAP (Promega) was added and each reaction was incubated at 30° C. At the time points indicated, 2 μl of reaction mixture was removed and assayed for luciferase expression as described below. All reactions were performed in triplicate.

Transfections: Lipoplexes were formed by mixing plasmid DNA with large unilamellar vesicles (LUVs) composed of equimolar amounts of DOPE:DODAC (50:50) on ice and incubated for 20 min prior to use. All transfections were performed at a cationic lipid to plasmid DNA charge ratio of 3:1. Lipoplexes were diluted with serum-containing media before addition directly to cell media. BHK cells were plated at 25,000 cells per well in 24-well plates. Neuro2A cells were plated at 30,000 cells per well in 24-well plates. The total mass of plasmid added was identical in all transfections. Equimolar transfections using plasmids of different sizes were achieved through the addition of an empty vector (pBlueScript) to normalize the total mass of DNA in each transfection. All transfections were performed in triplicate. Data is presented as mean values±standard error.

Luciferase and BCA Assays: Cells were washed twice with 1 mL PBS followed by the addition of 0.2 mL lysis buffer (PBS with 0.1% Triton X-100) before being stored at −70° C. Cells were thawed and 5-20 μl of sample were assayed in duplicate on a 96-well plate. Samples were assayed using a Berthhold Centro LB960 Microplate Luminometer and Luciferase Assay System (Promega). Standard luciferase assays were performed and transfection data is reported as mass quantities of luciferase protein using a standard curve obtained from serial 10-fold dilutions of a 20 mg/mL Photinus pyralis luciferase standard (Promega). Cell-free luciferase assays are reported in RLUs. Total protein was quantified using a Pierce BCA assay kit as per manufacturer's instructions.

Immunofluorescence Assays: BHK cells were plated on glass coverslips in 6-well plates (150,000 cells per well) and transfected with 1.5 μg of plasmid DNA. 24 h post-transfection, cells were washed once with 2 mL PBS-IF (10 mM sodium phosphate, 140 mM sodium chloride, pH 7.4) prior to fixation for 10 min with 2 mL 2% paraformaldehyde. Cells were subjected to three 30 s washes before permeabilization with 0.25% Triton X-100 in PBS-IF for 5 min. After washing three times for 30 s with PBS-IF, cells were incubated with blocking buffer (10% BSA in PBS-IF) for 1 h, shaking gently at room temperature. Cells were washed three times for 10 min with PBS-IF followed by addition of primary antibody solution comprising a 1:1000 dilution of goat anti-T7 RNAP antibody (a gift from Dr. Paul Fisher at the Department of Pharmacological Sciences, State University of New York at Stoney Brook) or 1:1000 dilution of mouse anti-luciferase monoclonal antibody (Abcam) in 2% BSA in PBS-IF. Cells were incubated with primary antibody solution for 2 h while shaking at room temperature. Cells were washed three times for 10 min in PBS-IF followed by the addition of secondary antibody (Rabbit anti-goat IgG, FITC labeled (QED Bioscience Inc) or Rabbit anti-mouse Texas Red labeled (Abcam), 1:200 dilution in 2% BSA-PBS-IF) and incubation for 2 h while shaking at room temperature. Cells were washed four times for 10 min with PBS-IF before being mounted and photographed on a Zeiss Axiovert S100 fluorescence microscope. Percentage of cells transfected was determined by counting transfected and non-transfected cells under the microscope. Data indicate the average of six separate counts from three different experiments.

RNase Protection Assay: RNAP and luciferase probes were prepared from EcoR 1 digested RPA-RNAP or RPA-Luc plasmid respectively. GAPDH probe was purchased from Pharmingen. Probes were labeled following the manufacturers protocol using 32P-αUTP (3000 Ci/mmole, 10 mCi/mL)(NEN).

BHK cells were plated on 6-well plates (150,000 cells per well) and transfected with 1.5 μg of R011 or L053 in triplicate. After 24 h cells were treated with 20 μg/mL Actinomycin D. At 0, 2, 4, 6 or 8 h after Actinomycin D treatments, cells were washed once with PBS and recovered by trypsinization. Cells from triplicate wells were pooled before harvesting total RNA (RNeasy miniprep kit, Qiagen). 10, 5 or 2.5 μg of total RNA was subjected to RNase protection analysis using the RiboQuant RPA system (Pharmingen) according to the manufacturers protocol. All values shown are the average±standard deviation of two independent experiments. Data was collected using a Typhoon Phosphoimager (Amersham Biosciences) and analysis was performed using ImageQuant software (Amersham Biosciences).

Primer Extension: Primer extension analysis using ³²P-labeled primer NVSC1 and 100 μg of RNA isolated from R011-transfected BHK cells (24 h post transfection) was performed using a Primer Extension System (Promega). The ladder was prepared by end labeling Φ X1 74 Hinf I DNA markers with ³²P. All values shown are the average±standard deviation of two independent experiments. Data was collected as described for RNase Protection assay above.

### Example 7

### Autocatalytic Gene Expression Results in an Exponential Time Dependent Increase in Gene Expression

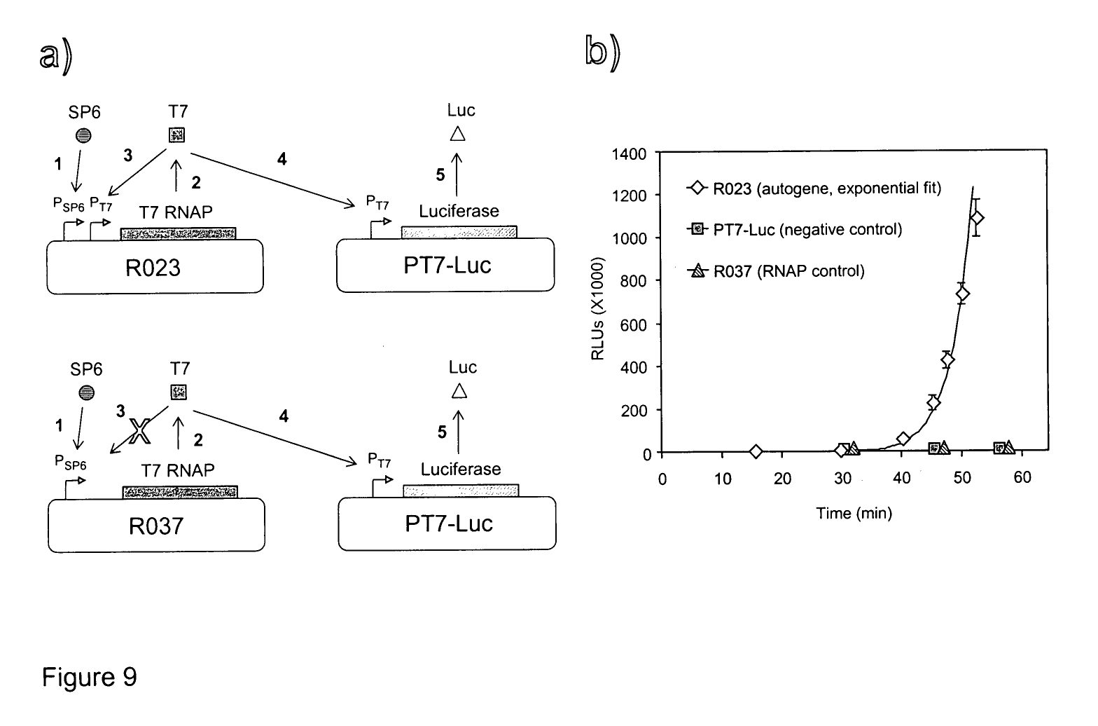

A hallmark of an autocatalytic, self-amplifying system is an exponential, time-dependent increase in the product being amplified. This exponential relationship would be limited only by the amount of substrate available (i.e. charged tRNA, GTP, etc.), and would continue as long as the template plasmid is in excess. In order to verify the autocatalytic nature of the autogene, a cell-free transcription and translation assay was performed. R023 plasmid DNA (comprising T7 RNAP driven by both SP6 and T7 RNAP promoters) was incubated with a PT7-Luc reporter gene plasmid (comprising luciferase driven by only the T7-promoter) in the presence of rabbit reticulocyte lysate and SP6 RNAP. SP6 RNAP transcribes T7 RNAP RNA from the R023 plasmid, leading to the production of T7 RNAP protein that is then able to drive expression of both the T7 RNAP gene from R023 in an autocatalytic fashion, as well as expression of the luciferase gene from PT7-Luc. FIG. 9 shows a dramatic increase in luciferase expression over time, indicating an exponential, autocatalytic increase in T7 RNAP protein. This increase is not observed when a control plasmid (R037, comprising T7 RNAP driven only by the SP6 promoter) lacking the T7 promoter needed for autocatalytic amplification is used. The reason for the lack of expression from R037 is that without the autocatalytic amplification, the amount of T7 RNAP produced is not enough to give rise to detectable levels of luciferase expression.

### Example 8

### A Bi-Cistronic Construct Results in Higher Levels of Gene Expression than a Dual Plasmid Transfection

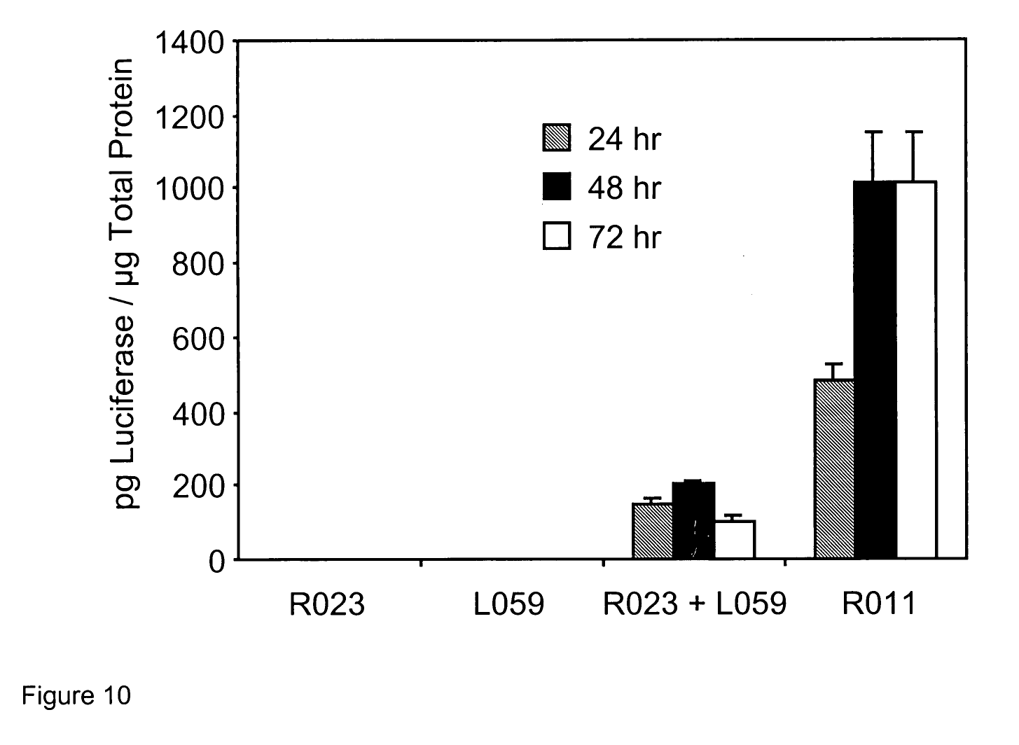

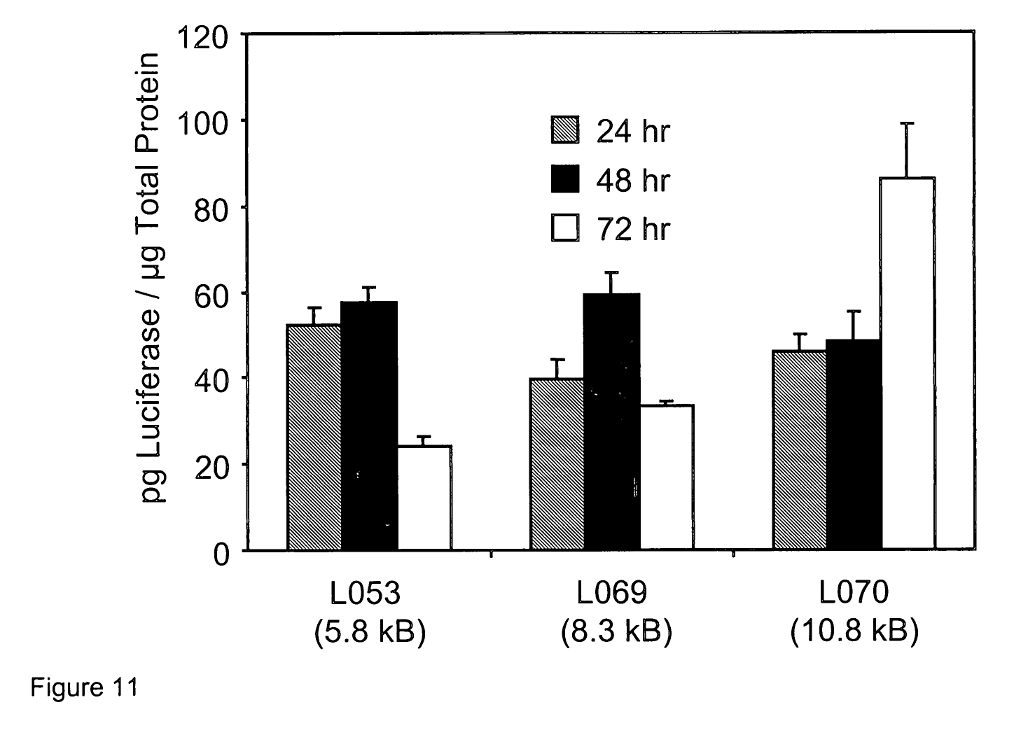

Previously published work on cytoplasmic expression systems employed an autogene cassette and a reporter gene cassette on separate plasmids. It was of interest to compare the expression resulting from a dual plasmid transfection system with a single plasmid bi-cistronic system in which the autogene and reporter gene were on one large plasmid. When equimolar amounts of autogene and reporter gene constructs were used to transfect BHK cells, it was found that the bi-cistronic construct yielded 2 to 4 fold higher levels of gene expression than the analogous dual plasmid transfection (FIG. 10). This result was unexpected because previous results suggest that transfection (delivery to nucleus and subsequent expression) would be more efficient for the smaller autogene plasmid than the larger bi-cistronic construct (see, e.g., Kreiss, et al Nucleic Acids Res. 27(19):3792-8 (1999)). For the dual plasmid transfection this would result in a greater number of cells expressing RNAP via the CMV promoter in the nucleus, and accordingly greater levels of luciferase via the RNAP promoter in the cytoplasm. In order to understand this phenomenon, a series of luciferase plasmids of increasing size were prepared to determine the effect of plasmid size on transfection efficiency in BHK cells. It was found that L053 (5.8 kb) L069 (8.3 kb) and L070 (10.8 kb) yielded similar levels of gene expression when transfected in equimolar amounts (FIG. 11). This suggests that for the system described here, larger plasmids are not at a disadvantage compared to the smaller plasmids. In addition, immunofluorescence studies using anti-T7 RNAP and anti-luciferase antibodies showed that the same percentage of cells are being transfected with the bi-cistronic construct as with the dual plasmid transfection.

### Example 9

### The Cytoplasmic Expression System Results in a 20-Fold Increase in Gene Expression Per Cell Compared to a Nuclear Expression System

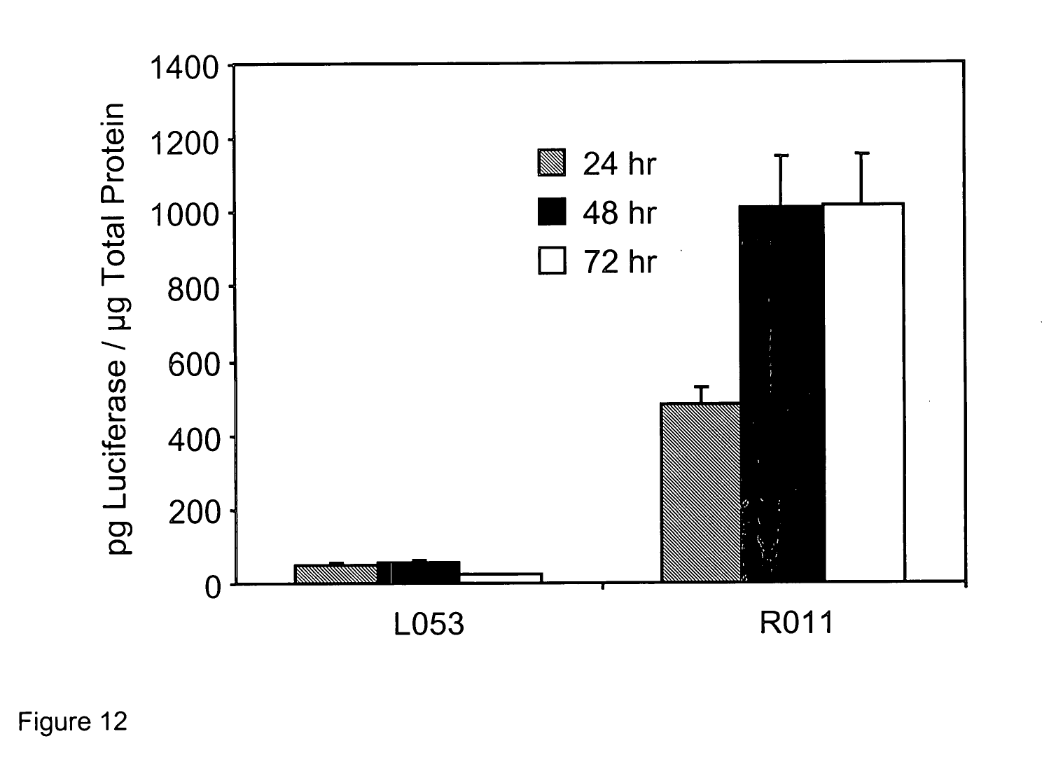

To compare the relative efficiency of nuclear versus cytoplasmic expression, BHK cells were transfected with equimolar amounts of a CMV-Luciferase (L053) and a bi-cistronic autogene plasmid containing both the autogene cassette, as well as the luciferase reporter gene cassette (R011). As shown in FIG. 12, the autogene system yielded a 20-fold increase in luciferase expression when compared with the CMV-mediated nuclear expression system.

To determine whether the increase in luciferase expression was the result of greater levels of luciferase production in each transfected cell or due to an increase in the total number of cells being transfected, the number of cells transfected with the autogene system was experimentally determined and compared with the number of cells transfected with the standard nuclear expression system. Transfected cells were quantified using immunofluorescence with both anti-T7-RNAP and anti-luciferase antibodies and BHK cells transfected with either the autogene or nuclear expression construct. The autogene and nuclear expression constructs both result in transfection of approximately the same number of cells (autogene 11.4%±3.5, nuclear 10.7%±2.9). The increase in reporter gene expression from the bi-cistronic autogene construct can therefore be attributed to an increase in the level of gene expression in transfected cells, as opposed to an increase in the number of cells being transfected.

The system described here is initially dependent on the nuclear transcription of T7 RNAP. As the two promoters have different transcription start sites, the two transcripts will have different length 5′-untranslated regions. To determine the proportion of nuclear transcripts derived from the CMV promoter versus cytoplasmic transcripts derived from the T7 promoter, a primer extension assay was performed using a primer that binds downstream of the two promoters, 90 bp downstream from PT7 and 300 bp downstream of the PCMV. A much higher proportion of mRNA is transcribed from the T7 promoter than from the CMV promoter (˜57±11 fold). This is consistent with previous work that found that the large majority of transcripts in the cell were transcribed by the T7 RNAP in the cytoplasm (see, e.g., Brisson, et al. Gene Ther. 6(2):263-270 (1999)) and further demonstrates that only a catalytic amount of RNAP needs to be expressed in the nucleus for large amounts of cytoplasmic mRNA to be produced.

### Example 10

### Cytoplasmic mRNA Transcripts have a Shorter Half-Life than Nuclear Transcripts

The lack of 5′ cap structure on the cytoplasmic transcripts would be expected to result in a decrease in mRNA stability (see, e.g., Drummond, et al. Nucleic Acids Res. 13(20):7375-94 (1985); Bernstein and Ross Trends Biochem. Sci. 14(9):373-7 (1989); Sachs Curr. Opin. Cell Biol. 2(6):1092-8 (1990); and Jackson and Standart Cell 62(1):15-24 (1990)). An RNAse Protection Assay (RPA) was used to measure both the half-life of the mRNA as well as the relative amounts of RNA present. BHK cells were transfected with equimolar amounts of R011 (autogene) and L053 (nuclear) plasmids. At 24 hours post-transfection, 20 μg/mL Actinomycin D was added to inhibit all de novo RNA synthesis. Previous work had demonstrated that this amount of Actinomycin D was sufficient to inhibit >99% of RNA synthesis. Cells were harvested at 2 hour intervals and total RNA was isolated. The half-life of the autogene transcripts average 103±6 min (88±3 min calculated using the RNAP probe, 115±5 min calculated using the Luciferase probe). The half-life of the nuclear transcripts was 317±6 min. By this analysis, we determined that the cytoplasmic transcripts are not as stable as the nuclear transcripts. Comparing the intensity of the luciferase transcript band from the nuclear and cytoplasmic transfections, there are approximately 20-fold more autogene-derived luciferase transcripts as there are nuclear luciferase transcripts. Given that the half-life of the autogene transcripts is three times shorter than the nuclear transcripts, these results suggest that the total output of the autogene system is at least 60 fold higher than the standard nuclear system.

### Example 11

### Autogene Expression is not Limited to BHK Cells

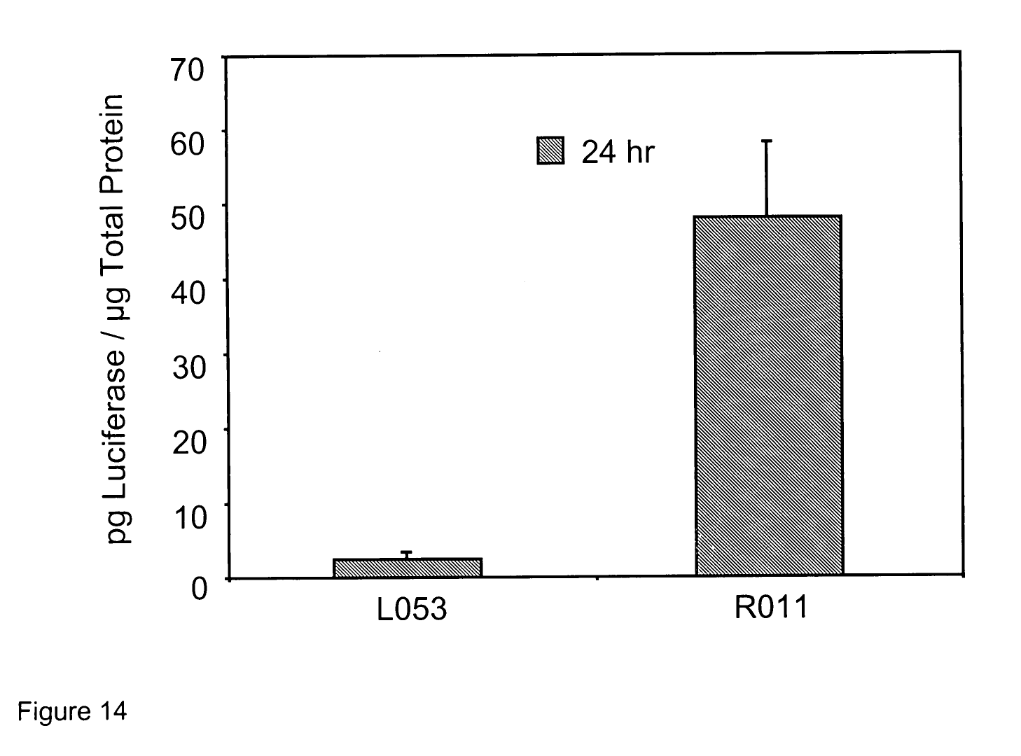

To determine whether the autogene effect seen with the BHK cells was specific to the cell line or if we could also achieve increases in gene expression in other cell lines, Neuro2A, a murine neuroblastoma cell line were transfected with R011 (autogene) or L053 (nuclear) and measured luciferase expression 24 h post transfection. As seen in FIG. 14, a 20-fold increase in gene expression is seen with the autogene when compared with the CMV-based nuclear expression construct. This indicates that the autogene expression previously seen is not limited to BHK cells alone.

### Example 12

### Summary

The bi-cistronic autogene system described here is distinguished from previously described systems. First, it contains both a CMV promoter, bypassing the need for addition of exogenous RNAP protein during transfection, as well as an autogene containing an EMCV IRES sequence, allowing for cap-independent translation of the autogene transcripts. In addition, our system has the autogene cassette and reporter gene cassette on the same plasmid, further simplifying the transfection process and resulting in increased transgene expression.

When we compared the expression levels from our cytoplasmic expression system and a standard nuclear expression system, the cytoplasmic system yielded 20-fold higher expression than the nuclear system. This is in contrast with previous systems that demonstrated a maximum of three-fold increase over a nuclear expression system control.11 The improvement in performance is most likely due to increased translation of cytoplasmic transcripts generated from our modified expression system. The inclusion of an EMCV IRES element in the autogene cassette described here appears to enhance translation, overcoming the lack of a 5′ cap on cytoplasmic transcripts and resulting in increased transgene expression levels.

We tested our autogene system in Neuro2A cells and also observed a 20-fold increase in expression with the autogene as compared with the CMV-based system. This indicates that the autogene system is not limited to BHK cells.

The mechanism whereby the bi-cistronic autogene system results in increased gene expression is of obvious interest. To verify that the T7 autogene does exhibit an autocatalytic expression profile. For the results described in FIG. 9, it is straightforward to show that if an autocatalytic process is occurring, then NL(t)=cet/τ, where NL(t) indicates the number of luciferase molecules at time t and c and τ are constants. The close fit (R2=0.94) of an exponential profile to the luciferase expression observed in FIG. 2 thus supports an autocatalytic mechanism. Any deviation from exponential characteristics at longer times can be attributed to either saturation effects as the amount of PT7-Luc becomes limiting, or the system running out of substrate (e.g. charged tRNA, GTP, etc).

The primer extension and RPA data provide further evidence of a cytoplasmic autocatalytic process. There is at least a 20-fold increase in transgene mRNA levels with the cytoplasmic expression system as compared to the standard nuclear expression system. These transcripts had a much shorter half-life than their nuclear counterparts, which is consistent with the lack of a 5′ cap, an important determinant of mRNA stability. When combined with the primer extension data showing that the majority of the transcripts are being made by the T7 RNAP, this data suggests that the increase in gene expression is due to an increase in mRNA levels in the cytoplasm of transfected cells, consistent with the autocatalytic process.

There are many possible explanations for why the bi-cistronic construct is more effective than a dual plasmid transfection. Without being bound by theory, one possible explanation is that the T7 RNAP is able to transcribe RNA from either the first PT7, driving T7 RNAP expression, or the second PT7, driving luciferase expression in the bi-cistronic construct. Due to the lack of terminator sequence between these two genes, both transcripts will encode for the luciferase gene. Therefore the cells transfected with the bi-cistronic plasmid should have more mRNA encoding luciferase than the cells in the dual transfection. Upon examination of the RPA data, it is clear that there are at least twice as many luciferase transcripts than RNAP transcripts following bi-cistronic transfection, lending support to this hypothesis. In addition, it was found that the luciferase transcripts had a slightly longer half-life than the T7 RNAP transcripts (115 min versus 88 min). This increased half-life may be attributed to the fact that the luciferase transcripts being made from the first PT7 in effect had a much longer 5′ UTR. This would most likely add some stability to the transcript, therefore increasing its half-life and subsequent luciferase expression.

The potential applications of an autogene based cytoplasmic expression system are many. Aside from increasing the levels of gene expression in plasmid-based non-viral gene delivery systems, this system can conveniently be used as a tool to express high levels of transgene in vitro for characterization or purification purposes.

In summary, the studies described here demonstrate a novel, bi-cistronic autogene based cytoplasmic expression system that shows 20-fold higher levels of gene expression compared with a nuclear expression system. This system has been shown to exhibit an exponential autocatalytic gene expression profile, and result in an increase in reporter gene expression per transfected cell, as opposed to an increase in the number of cells transfected. Furthermore, the bi-cistronic system has been demonstrated to be more effective than a cytoplasmic expression system carried on two plasmids. This system has a wide range of applications, not the least of which is increasing the therapeutic utility of plasmid based gene delivery systems.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An expression vector, said vector comprising an expression cassette comprising two components:
(a) a eukaryotic promoter and a first RNA polymerase promoter operably linked to a nucleic acid encoding a secretable RNA polymerase having a secretion domain, and a first internal ribosome entry site (IRES); and
(b) a second RNA polymerase promoter operably linked to a nucleic acid encoding a product of interest and a second internal ribosome entry site.

2. The expression vector of claim 1, wherein said eukaryotic promoter is a cytomegalovirus promoter.

3. The expression vector of claim 1, wherein said RNA polymerase is a non-host RNA polymerase.

4. The expression vector of claim 1, wherein said RNA polymerase is a T7 RNA polymerase.

5. The expression vector of claim 1, wherein said first IRES and said second IRES are the same.

6. The expression vector of claim 1, wherein said first IRES and said second IRES are different.

7. The expression vector of claim 1, wherein said first IRES and said second IRES are from encephalomyocarditisvirus.

8. The expression vector of claim 1, wherein said secretion domain is a member selected from the group consisting of: SEQ ID NOS: 1, 2, 3, 4, 5, 6, 7, 8, 9, 10, 11, 12, 13, 14, 15, 16, 17, 18, 19, 20, 21, 26, 27, 28, 29, 30, 31, 32, 33, 34, 35, 36, 37, 38, 39, 40, 41, 42, 43, 44, and 45.

9. The expression vector of claim 1, wherein said product of interest is a therapeutic product.

10. The expression vector of claim 9, wherein said therapeutic product is a member selected from the group consisting of: a protein, a nucleic acid, an antisense nucleic acid, ribozymes, tRNA, siRNA, and an antigen.

11. A host cell comprising the expression vector of claim 1.

12. A lipid-nucleic acid composition comprising:
a nucleic acid-lipid particle comprising a lipid portion and a nucleic acid portion,
wherein said nucleic acid portion comprises an expression cassette comprising two components:
(a) a eukaryotic promoter and a first RNA polymerase promoter operably linked to a nucleic acid encoding a secretable RNA polymerase having a secretion domain, and a first internal ribosome entry site; and
(b) a second RNA polymerase promoter operably linked to a nucleic acid encoding a product of interest and a second internal ribosome entry site.

13. The lipid-nucleic acid composition of claim 12, wherein said nucleic acid-lipid particle is a serum-stable nucleic acid-lipid particle comprising a nucleic acid fully encapsulated within said lipid portion.

14. The lipid-nucleic acid composition of claim 12, wherein said lipid portion comprises a cationic lipid, a non-cationic lipid; and a polyethyleneglycol-lipid conjugate.

15. The lipid-nucleic acid composition of claim 14, wherein said cationic lipid is a member selected from the group consisting of: N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and a mixture thereof.

16. The lipid-nucleic acid composition of claim 14, wherein said non-cationic lipid is a member selected from the group consisting of dioleoylphosphatidylethanolamine (DOPE), palmitoyloleoylphosphatidylcholine (POPC), egg phosphatidylcholine (EPC), distearoylphosphatidylcholine (DSPC), cholesterol, and a mixture thereof.

17. The lipid-nucleic acid composition of claim 14, wherein said cationic lipid comprises from about 2% to about 60% of the total lipid present in said particle.

18. The lipid-nucleic acid composition of claim 14, wherein said non-cationic lipid comprises from about 5% to about 90% of the total lipid present in said particle.

19. The lipid-nucleic acid composition of claim 14, wherein said PEG-lipid conjugate comprises from 1% to about 20% of the total lipid present in said particle.

20. The lipid-nucleic acid composition of claim 14, wherein said non-cationic lipid is DSPC.

21. The lipid-nucleic acid composition of claim 14, further comprising cholesterol.

22. The lipid-nucleic acid composition of claim 21, wherein the cholesterol comprises from about 10% to about 60% of the total lipid present in said particle.

23. The lipid-nucleic acid composition of claim 14, wherein
the cationic lipid comprises 7.5% of the total lipid present in said particle;
the non-cationic lipid comprises 82.5% of the total lipid present in said particle; and
the PEG- lipid conjugate comprises 10% of the total lipid present in said particle.

24. The lipid-nucleic acid composition of claim 14, wherein the nucleic acid-lipid particle comprises:
DODMA;
DSPC; and
a PEG-lipid conjugate.

25. The lipid-nucleic acid composition of claim 24, further comprising cholesterol.

26. A method of expressing a nucleic acid encoding a product of interest in a cell, said method comprising:
introducing into a cell an expression vector comprising an expression cassette comprising two components:
(a) a eukaryotic promoter and a first RNA polymerase promoter operably linked to a nucleic acid encoding a secretable RNA polymerase having a secretion domain, and a first internal ribosome entry site; and
(b) a second RNA polymerase promoter operably linked to a nucleic acid encoding a product of interest and a second internal ribosome entry site.

27. The method of claim 26, wherein said RNA polymerase is a T7 RNA polymerase.

28. The method of claim 26, wherein said secretion domain is a member selected from the group consisting of: SEQ ID NOS: 1, 2, 3, 4, 5, 6, 7, 8, 9, 10, 11, 12, 13, 14, 15, 16, 17, 18, 19, 20, 21, 26, 27, 28, 29, 30, 31, 32, 33, 34, 35, 36, 37, 38, 39, 40, 41, 42, 43, 44, and 45.

29. The method of claim 26, wherein said expression vector is fully encapsulated in a lipid portion of a serum stable nucleic acid-lipid particle.

30. The method of claim 26, wherein said product of interest is a therapeutic product.

31. The method of claim 26, wherein said therapeutic product is a member selected from the group consisting of: a protein, a nucleic acid, an antisense nucleic acid, ribozymes, tRNA, siRNA, and an antigen.

32. A method of delivering a nucleic acid encoding a product of interest to a cell, said method comprising:
introducing into the cell an expression vector comprising an expression cassette comprising two components:
(a) a eukaryotic promoter and a first RNA polymerase promoter operably linked to a nucleic acid encoding a secretable RNA polymerase having a secretion domain, and a first internal ribosome entry site; and
(b) a second RNA polymerase promoter operably linked to a nucleic acid encoding a product of interest and a second internal ribosome entry site.

33. The method of claim 32, wherein said cell is in a mammal.

34. The method of claim 33, wherein said mammal is a human.

35. A method of treating a disease in a subject, comprising:
administering a therapeutically effective amount of an expression cassette comprising two components:
(a) a eukaryotic promoter and a first RNA polymerase promoter operably linked to a nucleic acid encoding a secretable RNA polymerase having a secretion domain, and a first internal ribosome entry site; and
(b) a second RNA polymerase promoter operably linked to a nucleic acid encoding a therapeutic product and a second internal ribosome entry site.

36. The method of claim 35, wherein said subject is a mammal.

37. The method of claim 36, wherein said mammal is a human.

38. The method of claim 35, wherein said expression vector is fully encapsulated in a lipid portion of a serum stable nucleic acid-lipid particle.

39. The method of claim 35, wherein said disease is a member selected from the group consisting of: a cancer, an autoimmune disease, a cardiovascular disease, a viral disease, a bacterial disease, and an inflammatory disease.

40. An isolated purified nucleic acid comprising the sequence set forth in SEQ ID NO: 46, 50, or 51.
